(19) United States
(12) Patent Application Publication
(10) Pub. No.: US 2023/0102247 A1
(43) Pub. Date: Mar. 30, 2023

(54) METHODS AND COMPOSITIONS RELATING TO IONIC LIQUID ADJUVANTS

(71) Applicant: PRESIDENT AND FELLOWS OF HARVARD COLLEGE, Cambridge, MA (US)

(72) Inventors: Samir MITRAGOTRI, Cambridge, MA (US); Anvay Ashish UKIDVE, Cambridge, MA (US); Katharina CU, Cambridge, MA (US)

(73) Assignee: PRESIDENT AND FELLOWS OF HARVARD COLLEGE, Cambridge, MA (US)

(21) Appl. No.: 17/921,158

(22) PCT Filed: Apr. 27, 2021
(86) PCT No.: PCT/US2021/029326

§ 371 Date: Oct. 25, 2022

Related U.S. Application Data
(60) Provisional application No. 63/016,360, filed on Apr. 28, 2020.

(51) Int. Cl. A61K 39/39; A61K 39/215; A61K 39/145; A61K 39/29; A61K 39/00

(52) U.S. Cl. CPC A61K 39/39; A61K 39/215; A61K 39/145; A61K 39/292; A61K 39/0016; A61K 2039/55511

(57) ABSTRACT

The technology described herein is directed to adjuvants comprising ionic liquids, as well as compositions and methods utilizing or comprising such adjuvants.

### CROSS-REFERENCE TO RELATED APPLICATIONS

This application claims benefit under 35 U.S.C. § 119(e) of U.S. Provisional Application No. 63/016,360 filed Apr. 28, 2020, the contents of which are incorporated herein by reference in their entirety.

### TECHNICAL FIELD

The technology described herein relates to compositions and methods relating to adjuvants, e.g, for vaccination.

### BACKGROUND

Adjuvants form an important, and often essential, component of effective vaccines by serving to stimulate immune responses so that a protective and long-lasting immunological memory of the antigen is created. While several materials have been explored for use as adjuvants, although only a few including aluminum salts (alum), bacterial lipids (monophosphoryl A) and foreign genome (CpG) are commonly used. A key reason for this limited development of adjuvants is the safety concern. Design of potent and safe adjuvants poses a significant challenge since they must strike a delicate balance between strong local immune stimulation and low systemic toxicity. Development of new adjuvants is a key aspect of addressing infectious diseases in the future.

### SUMMARY

Described herein is the finding that ionic liquids (e.g., CoLa) are safe and effective adjuvants. It is demonstrated that this new class of adjuvants distributes the antigen efficiently upon injection, maintains antigen integrity, enhances immune infiltration at the injection site, and leads to a potent immune response against the antigen.

In particular, the use of ionic liquid adjuvants is demonstrated to induce both Th1 and Th2 responses, thought the Th1 reponses was more strongly induced. The Th1 response observed included increases in dendritic, NK, CD4+, and CD8+ cells, with increased infiltration of CD4+(but not CD8+ cells) and dendritic cells into the site of immunization. The dendritic cells also displayed markers for increased activation. No toxicity was observed. CoLa and ionic liquids in general, provide a notable addition to the repertoire of available adjuvants for addressing unmet needs for protection against pandemics like COVID-19 and future infectious agent threats.

In one aspect of any of the embodiments, described herein is a method of immunizing a subject, the method comprising administering to the subject i) an adjuvant comprising an ionic liquid; and ii) at least one antigen. In one aspect of any of the embodiments, described herein is a method of immunizing a subject, the method comprising administering to the subject a composition comprising i) an adjuvant comprising an ionic liquid; and ii) at least one antigen. In one aspect of any of the embodiments, described herein is a method of stimulating an immune response of a subject, the method comprising administering to the human an adjuvant comprising an ionic liquid. In one aspect of any of the embodiments, described herein is a vaccine composition comprising: an adjuvant comprising an ionic liquid; and at least one antigen. In one aspect of any of the embodiments, described herein is a vaccine composition comprising: an adjuvant comprising an ionic liquid; and at least one antigen; for use in a method of immunizing a subject and/or stimulating an immune response of a subject.

In some embodiments of any of the aspects, the immune response is, or the administration results in an immune response which is a Th1 and/or Th2 response. In some embodiments of any of the aspects, the immune response is, or the administration results in an immune response which is an increase in Th1 and/or Th2 response as compared to the level in the absence of the adjuvant. In some embodiments of any of the aspects, the immune response is, or the administration results in an immune response which is an increase in Th1 response as compared to the level in the absence of the adjuvant. In some embodiments of any of the aspects, the immune response is, or the administration results in an immune response which is, an increase in activation and/or infiltration of dendritic cells as compared to the level in the absence of the adjuvant. In some embodiments of any of the aspects, the immune response is, or the administration results in an immune response which is, an increase in the number and/or infiltration of CD4+ cells as compared to the level in the absence of the adjuvant. In some embodiments of any of the aspects, the immune response is, or the administration results in an immune response which is, an increase in the number of NK and/or CD8+ cells as compared to the level in the absence of the adjuvant.

In some embodiments of any of the aspects, the administration is by injection, subcutaneous injection, or mucosal administration. In some embodiments of any of the aspects, the administration of the adjuvant and antigen causes a greater immune response, increased rate of an immune response, and/or greater protection than the same dose of the antigen administered without the adjuvant. In some embodiments of any of the aspects, a therapeutically effective dose(s) is/are administered. In some embodiments of any of the aspects, a therapeutically effective dose of the adjuvant and antigen comprises less antigen than a therapeutically effective dose of the antigen in the absence of the adjuvant.

In some embodiments of any of the aspects, the ionic liquid comprises a quaternary ammonium cation. In some embodiments of any of the aspects, the ionic liquid comprises a choline cation.

In some embodiments of any of the aspects, the ionic liquid comprises an organic acid anion. In some embodiments of any of the aspects, the ionic liquid comprises an organic acid anion with a log P of less than one. In some embodiments of any of the aspects, the ionic liquid comprises a lactic acid anion.

In some embodiments of any of the aspects, the ionic liquid is choline:lactic acid (CoLa).

In some embodiments of any of the aspects, the ionic liquid is at a concentration of from 1%-50% w/v. In some embodiments of any of the aspects, the ionic liquid is at a concentration of from 1%-30% w/v. In some embodiments of any of the aspects, the ionic liquid is at a concentration of from 5%-20% w/v. In some embodiments of any of the aspects, the ionic liquid is at a concentration of 10% w/v. In some embodiments of any of the aspects, the ionic liquid is an emulsion in saline. In some embodiments of any of the aspects, the ionic liquid has a cation:anion molar ratio of from 1:1 to 1:4. In some embodiments of any of the aspects, the ionic liquid has a cation:anion molar ratio of 1:2.

In some embodiments of any of the aspects, the antigen is comprised by a vaccine selected from the group consisting of: a coronavirus vaccine; a SARS-CoV-2 vaccine; a pneumococcal vaccine; an influenza vaccine; a hepatitis B (HBV) vaccine; an acellular pertussis (aP) vaccine; a diphtheria tetanus acellular pertussis (DTaP) vaccine; a hepatitis A (HAV) vaccine; and a meningococcal (MV) vaccine. In some embodiments of any of the aspects, the antigen is a molecule or motif obtained or derived from: a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus.

### BRIEF DESCRIPTION OF THE DRAWINGS

FIGS. 1A-1G demonstrate that CoLa adsorbs, disperses and withholds the release of OVA while maintaining integrity. FIG. 1A. ¹H NMR spectrum of Choline lactate (1:2). FIG. 1B. Percent of incubated OVA adsorbed on adjuvants (n=3 for all groups). FIG. 1C. Percent cumulative release of adsorbed OVA from two adjuvants (n=3 for all groups). FIG. 1D. Fluorescence images of porcine skin dispersion of fluorescently labeled OVA for CoLa and alum. Scale bar: 1000 μm. FIG. 1E. Quantitative area coverage of dispersed OVA in mm² (n=6 for both groups). *: p<0.05. Significantly different. (Unpaired t-test) FIG. 1F. SDS-PAGE analysis of OVA with different adjuvants showing a distinct band between 37-50 kDa, indicating stable OVA. FIG. 1G. Circular dichroism spectra demonstrating conserved a helices for all adjuvants. Data in FIGS. 1B, 1C, and 1E are represented as mean±s.e.m.

FIGS. 2A-2H demonstrate that CoLa improves immune infiltration at the injection site leading to potent systemic Th1 immune responses. FIG. 2A. Quantitative analysis of infiltrating CD11c+ of CD45+ cells (n=4 for all groups) at the injection site. FIG. 2B. Quantitative analysis of median fluorescent intensity of CD 86 on CD11c+ cells at the injection site (n=4 for all groups). FIG. 2C. Quantitative analysis of infiltrating CD4+ of CD45+ cells at the injection site (n=4 for all groups). FIG. 2D. Anti-OVA IgG antibody titer for different adjuvants (n=7 for all groups). FIG. 2E. Quantitative analysis of CD8+CD3+ of CD45+ cells (n=8 for all groups) in spleen. FIG. 2F. Quantitative analysis of NKp46+ of CD45+ cells (n=8 for all groups) in spleen. FIG. 2G. Quantitative analysis of CD11c+ of CD45+ cells (n=8 for all groups) in spleen. Orange rectangle indicates further analysis of median fluorescent intensity of CD80 for CoLa and Alum. FIG. 2H. Quantitative analysis of CD4+CD3+ of CD45+ cells (n=8 for all groups) in spleen. Green rectangle indicates further analysis of IFN-γ+CD4+ cells for CoLa and Alum. For FIGS. 2A-2C, UnTx indicates untreated mice. For FIGS. 2A-2H, Significantly different. *: p<0.05, **: p<0.01 (One-way ANOVA followed by Tukey's HSD). For orange and green rectangles in FIGS. 2G and 2H, significantly different. *: p<0.05 (Unpaired t-test). Data in FIGS. 2A-2H are represented as mean±s.e.m.

FIG. 3. Schematic of developmental plan for CoLa as an adjuvant

FIG. 4. Percentage protein adsorption as a function of protein added to the adjuvant formulation.

FIG. 5. Illustration of depth and width of dispersed antigen from fluorescent images were used to identify the region of interest (ROI). A MATLAB code was used to determine the area of dispersion within that ROI.

FIGS. 6A-6B. Effect of increasing CoLa concentration in adjuvant formulation on dispersion. FIG. 6A. Width of spread in mm. FIG. 6B. Depth of spread in mm. (n=6 for all groups). *: p<0.05, **: p<0.01. Significantly different. One-way ANOVA followed by Tukey's HSD test.

FIG. 7. Representative flow cytometry graphs for infiltrating CD45+CD11c+ cells at the injection site

FIG. 8. Depicts auantitative analysis of infiltrated CD8+ cells from CD45+ cells at the site of injection 24 h after adjuvant administration.

FIGS. 9A-9B depict the schedule and toxicity assessment of CoLa vaccination. FIG. 9A. Schedule for vaccination and organ harvesting. FIG. 9B. Percent change in body weight for different treatment groups. Different treatment groups show no effect on weight change (n=8 for all groups) (Two-way ANOVA followed by Tukey

### DETAILED DESCRIPTION

In one aspect of any of the embodiments, described herein is a method of immunizing a subject, the method comprising administering to the subject i) an adjuvant comprising an ionic liquid; and ii) at least one antigen. In one aspect of any of the embodiments, described herein is a method of method of stimulating an immune response of a subject, the method comprising administering to the human an adjuvant comprising an ionic liquid. In one aspect of any of the embodiments, described herein is a vaccine composition comprising: i) an adjuvant comprising at least one ionic liquid; and ii) at least one antigen.

The terms “immunize” and “vaccinate” tend to be used interchangeably in the field. However, in reference to the administration of the vaccine compositions as described herein to provide protection against disease, e.g., infectious disease caused by a pathogen, it should be understood that “vaccinate” refers to the administration of a vaccine composition and the term “immunize” refers to the process of conferring, increasing, or inducing the passive protection conferred by the administered vaccine composition.

As used herein in the context of immunization, immune response and vaccination, the term “adjuvant” refers to any substance than when used in combination with a specific antigen that produces a more robust immune response than the antigen alone. When incorporated into a vaccine formulation, an adjuvant acts generally to accelerate, prolong, or enhance the quality of specific immune responses to the vaccine antigen(s).

The adjuvants described herein can comprise one or more ionic liquids. The term “ionic liquids (ILs)” as used herein refers to organic salts or mixtures of organic salts which are in liquid state at room temperature. This class of solvents has been shown to be useful in a variety of fields, including in industrial processing, catalysis, pharmaceuticals, and electrochemistry. The ionic liquids contain at least one anionic and at least one cationic component. Ionic liquids can comprise an additional hydrogen bond donor (i.e. any molecule that can provide an —OH or an —NH group), examples include but are not limited to alcohols, fatty acids, and amines. The at least one anionic and at least one cationic component may be present in any molar ratio. Exemplary molar ratios (cation:anion) include but are not limited to 1:1, 1:2, 2:1, 1:3, 3:1, 2:3, 3:2, and ranges between these ratios. For further discussion of ionic liquids, see, e.g., Hough, et ah, “The third evolution of ionic liquids: active pharmaceutical ingredients”, New Journal of Chemistry, 31: 1429 (2007) and Xu, et al., “Ionic Liquids: Ion Mobilities, Glass Temperatures, and Fragilities”, Journal of Physical Chemistry B, 107(25): 6170-6178 (2003); each of which is incorporated by reference herein in its entirety. In some embodiments of any of the aspects, the ionic liquid or solvent exists as a liquid below 100° C. In some embodiments of any of the aspects, the ionic liquid or solvent exists as a liquid at room temperature.

Choline and derivatives thereof are particularly well suited as IL cations for the ionic liquids described herein. Accordingly, the cation of an IL described herein can be a cation comprising a quaternary ammonium. A quarternary ammonion is a positively charged polyatomic ion of the structure NR₄⁺, each R independently being an alkyl group or an aryl group.

The general term “quaternary ammonium” relates to any compound that can be regarded as derived from ammonium hydroxide or an ammonium salt by replacement of all four hydrogen atoms of the NH₄⁺ ion by organic groups. For example, the quaternary ammonium has the structure of NR₄⁺, where each R is independently selected from hydroxyl, optionally substituted C₁-C₁₀alkyl, optionally substituted C₂-C₁₀alkenyl, optionally substituted C₂-C₁₀alkynyl, optionally substituted aryl, or optionally substituted heteroaryl.

In some embodiments of any of the aspects, the cation has a molar mass equal to or greater than choline, e.g., a molar mass equal to or greater than 104.1708 g/mol. In some embodiments of any of the aspects, the cation has a molar mass greater than choline, e.g., a molar mass equal greater than 104.1708 g/mol.

In some embodiments of any of the aspects, each R group of the quaternary ammonium independently comprises an alkyl, alkane, alkene, or aryl. In some embodiments of any of the aspects, each R group of the quaternary ammonium independently comprises an alkyl, alkane, or alkene. In some embodiments of any of the aspects, each R group of the quaternary ammonium independently comprises an alkane or alkene. In some embodiments of any of the aspects, each R group of the quaternary ammonium independently comprises a carbon chain of no more than 10 carbon atoms in length, e.g., no more than 10, 11, 12, 13, 14, 15, 16, 17, 18, 19, 20, 25, or 30 carbon atoms in length. In some embodiments of any of the aspects, each R group of the quaternary ammonium independently comprises a carbon chain of no more than 12 carbon atoms in length. In some embodiments of any of the aspects, each R group of the quaternary ammonium independently comprises a carbon chain of no more than 15 carbon atoms in length. In some embodiments of any of the aspects, each R group of the quaternary ammonium independently comprises a carbon chain of no more than 20 carbon atoms in length.

In some embodiments of any of the aspects, each R group of the quaternary ammonium independently comprises a carbon chain of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternary ammonium independently comprises a carbon chain of no more than 12 carbon atoms. In some embodiments of any of the aspects, each R group of the quaternary ammonium independently comprises a carbon chain of no more than 15 carbon atoms. In some embodiments of any of the aspects, each R group of the quaternary ammonium independently comprises a carbon chain of no more than 20 carbon atoms.

In some embodiments of any of the aspects, each R group of the quaternary ammonium independently comprises an alkyl group of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternary ammonium independently comprises an alkyl group of no more than 12 carbon atoms. In some embodiments of any of the aspects, each R group of the quaternary ammonium independently comprises an alkyl group of no more than 15 carbon atoms. In some embodiments of any of the aspects, each R group of the quaternary ammonium independently comprises an alkyl group of no more than 20 carbon atoms.

In some embodiments of any of the aspects, each R group of the quaternary ammonium independently comprises an alkane, alkene, aryl, heteroaryl, alkyl, or heteroalkyl. In some embodiments of any of the aspects, each R group of the quaternary ammonium independently comprises an unsubstituted alkane, unsubstituted alkene, unsubstituted aryl, unsubstituted heteroaryl, unsubstituted alkyl, or unsubstituted heteroalkyl. In some embodiments of any of the aspects, each R group of the quaternary ammonium independently comprises an unsubstituted alkane. In some embodiments of any of the aspects, each R group of the quaternary ammonium independently comprises an unsubstituted alkene. In some embodiments of any of the aspects, each R group of the quaternary ammonium independently comprises one or more substituent groups.

In some embodiments of any of the aspects, at least one R group of the quaternary ammonium comprises a hydroxy group. In some embodiments of any of the aspects, one R group of the quaternary ammonium comprises a hydroxy group. In some embodiments of any of the aspects, only one R group of the quaternary ammonium comprises a hydroxy group.

Exemplary, non-limiting cations can include choline and any of the cations designated C1-C7 which are defined by structure below.

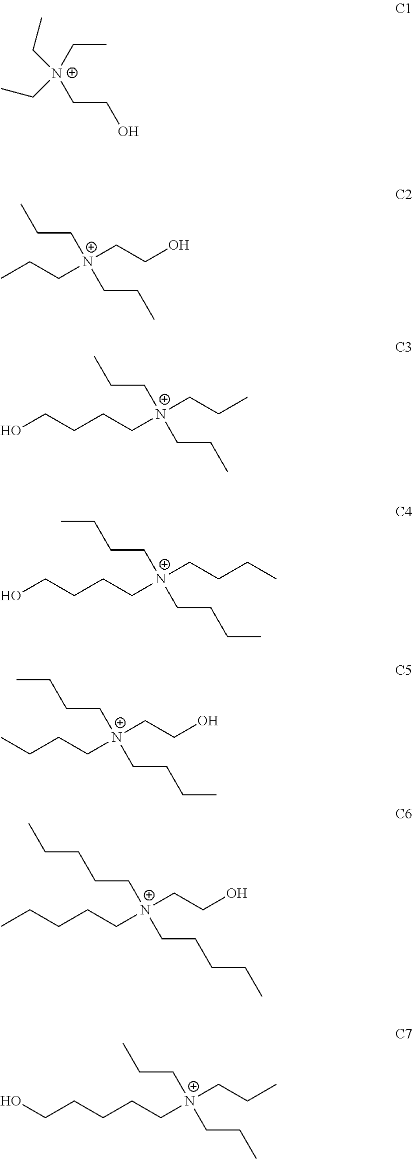

Further non-limiting examples of cations include the following:
1-(hydroxymethyl)-1-methylpyrrolidin-1-ium
1-(2-hydroxyethyl)-1-methylpyrrolidin-1-ium
1-ethyl-1-(3-hydroxypropyl)pyrrolidin-1-ium
1-(3-hydroxypropyl)-1-methylpyrrolidin-1-ium
1-(4-hydroxybutyl)-1-methylpyrrolidin-1-ium
1-ethyl-1-(4-hydroxybutyl)pyrrolidin-1-ium
1-(4-hydroxybutyl)-1-propylpyrrolidin-1-ium
1-(5-hydroxypentyl)-1-propylpyrrolidin-1-ium
1-ethyl-1-(5-hydroxypentyl)pyrrolidin-1-ium
1-(5-hydroxypentyl)-1-methylpyrrolidin-1-ium
1-(hydroxymethyl)-1-methylpiperidin-1-ium
1-(2-hydroxyethyl)-1-methylpiperidin-1-ium
1-ethyl-1-(2-hydroxyethyl)piperidin-1-ium
1-ethyl-1-(3-hydroxypropyl)piperidin-1-ium
1-(3-hydroxypropyl)-1-propylpiperidin-1-ium
1-(3-hydroxypropyl)-1-methylpiperidin-1-ium
1-(4-hydroxybutyl)-1-methylpiperidin-1-ium
1-ethyl-1-(4-hydroxybutyl)piperidin-1-ium
1-(4-hydroxybutyl)-1-propylpiperidin-1-ium
1-butyl-1-(5-hydroxypentyl)piperidin-1-ium
1-(5-hydroxypentyl)-1-propylpiperidin-1-ium
1-ethyl-1-(5-hydroxypentyl)piperidin-1-ium
1-(5-hydroxypentyl)-1-methylpiperidin-1-ium
3-ethyl-1-methyl-1H-imidazol-3-ium
1-methyl-3-propyl-1H-imidazol-3-ium
3-butyl-1-methyl-1H-imidazol-3-ium
1-methyl-3-pentyl-1H-imidazol-3-ium
1,2-dimethyl-3-pentyl-1H-imidazol-3-ium
3-butyl-1,2-dimethyl-1H-imidazol-3-ium
1,2-dimethyl-3-propyl-1H-imidazol-3-ium
3-(hydroxymethyl)-1,2-dimethyl-1H-imidazol-3-ium
3-(2-hydroxyethyl)-1,2-dimethyl-1H-imidazol-3-ium
3-(3-hydroxypropyl)-1,2-dimethyl-1H-imidazol-3-ium
3-(4-hydroxybutyl)-1,2-dimethyl-1H-imidazol-3-ium
3-(5-hydroxypentyl)-1,2-dimethyl-1H-imidazol-3-ium
3-(5-hydroxypentyl)-1-methyl-1H-imidazol-3-ium
3-(4-hydroxybutyl)-1-methyl-1H-imidazol-3-ium
3-(3-hydroxypropyl)-1-methyl-1H-imidazol-3-ium
3-(2-hydroxyethyl)-1-methyl-1H-imidazol-3-ium
3-(hydroxymethyl)-1,2,4,5-tetramethyl-1H-imidazol-3-ium
3-(2-hydroxyethyl)-1,2,4,5-tetramethyl-1H-imidazol-3-ium
3-(3-hydroxypropyl)-1,2,4,5-tetramethyl-1H-imidazol-3-ium
3-(4-hydroxybutyl)-1,2,4,5-tetramethyl-1H-imidazol-3-ium
3-(5-hydroxypentyl)-1,2,4,5-tetramethyl-1H-imidazol-3-ium
1-(5-hydroxypentyl)pyridin-1-ium
1-(4-hydroxybutyl)pyridin-1-ium
1-(3-hydroxypropyl)pyridin-1-ium
1-(2-hydroxyethyl)pyridin-1-ium
1-(hydroxymethyl)pyridin-1-ium
1-hydroxypyridin-1-ium
(hydroxymethyl)trimethylphosphonium
triethyl(hydroxymethyl)phosphonium
triethyl(2-hydroxyethyl)phosphonium
(2-hydroxyethyl)tripropylphosphonium
(3-hydroxypropyl)tripropylphosphonium
tributyl(3-hydroxypropyl)phosphonium
(3-hydroxypropyl)tripentylphosphonium
(4-hydroxybutyl)tripentylphosphonium
(5-hydroxypentyl)tripentylphosphonium

In some embodiments of any of the aspects, the cation is choline, C1, C6, and/or C7. In some embodiments of any of the aspects, the cation is C1, C6, and/or C7. In some embodiments of any of the aspects, the cation is choline.

Anions with low hydrophobicity or relatively short carbon chains provide improved performance as adjuvants. In some embodiments of any of the aspects, the anion of an IL described herein is hydrophobic.

In some embodiments of any of the aspects, the anion of an IL described herein is an organic acid. In some embodiments of any of the aspects, the anion of an IL described herein comprises a carboxylic acid. In some embodiments of any of the aspects, the anion of an IL described herein comprises a carboxylic acid which is not a fatty acid.

A carboxylic acid is a compound having the structure of Formula I, wherein R can be any group.

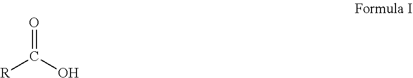

Generally, the anion is R—X⁻, where X is CO₂⁻, SO₃⁻, OSO₃²⁻ or OPO₃²⁻; and R is optionally substituted C₁-C₁₀alkyl, optionally substituted C₂-C₁₀alkenyl, or optionally substituted C₂-C₁₀alkynyl, optionally substituted aryl, or optionally substituted heteroaryl.

In some embodiments, R is an optionally substituted linear or branched C₁-C₉alkyl. For example, R is a C₁-C₉alkyl optionally substituted with 1, 2, 3, 4, 5 or 6 substituents independently selected from the group consisting of C₁-C₃alkyl, hydroxy (OH), halogen, oxo (═O), carboxy (CO₂), cyano (CN) and aryl. In some embodiments, R is a C₁-C₆alkyl optionally substituted with 1, 2, 3, 4 or 5 substituents independently selected from the group consisting of C₁-C₃alkyl, hydroxy, carboxy and phenyl. Preferably, R is a C₁-C₅alkyl, optionally substituted with 1, 2, 3, 4 or 5 substituents independently selected from the group consisting of methyl, ethyl, hydroxyl, carboxy, and phenyl. Exemplary alkyls for R include, but are not limited to, methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl and nonyl.

In some embodiments, R is an optionally substituted linear or branched C₂-C₅alkenyl. For example, R is a C₂-C₉alkenyl optionally substituted with 1, 2, 3, 4, 5 or 6 substituents independently selected from the group consisting of C₁-C₃alkyl, hydroxy, halogen, oxo, carboxy, cyano and aryl. In some embodiments, R is a C₂-C₅alkenyl optionally substituted with 1, 2, 3, 4 or 5 substituents independently selected from the group consisting of C1-C₃alkyl, hydroxy, carboxy and phenyl. Preferably, R is a C₁-C₅alkenyl, optionally substituted with 1, 2, 3, 4 or 5 substituents independently selected from the group consisting of methyl, ethyl, hydroxyl, carboxy, and phenyl. Exemplary alkenyls for R include, but are not limited to, ethenyl, 2-carboxyethenyl, 1-methylpropenyl and 2-methylpropenyl.

In some embodiments, R is an optionally substituted aryl or heteroaryl. For example, R is an aryl or heteroayl optionally substituted with 1, 2, 3, 4, 5 or 6 substituents independently selected from the group consisting of C₁-C₃alkyl, hydroxy, halogen, oxo, carboxy, cyano and aryl. In some embodiments, R is an aryl optionally substituted with 1, 2, 3, 4 or 5 substituents independently selected from the group consisting of C₁-C₃alkyl, hydroxy, carboxy and phenyl. Preferably R is a phenyl substituted with 1, 2 or 3 substituents independently selected from the group consisting of methyl, ethyl, hydroxyl, carboxy, and phenyl. Exemplary aryls for R include, but are not limited to, phenyl, 2-hydroxyphenyl, 3-hydroxyphenyl, 4-hydroxyphenyl, dihydroxyphenyl, trihydroxyphenyl, 3,4,5-trihydroxyphenyl, and 1,1-biphen-4-yl.

In some embodiments, X is CO₂⁻ and R is methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl, nonyl, ethenyl, 2-carboxyethenyl, 1-methylpropenyl, 2-methylpropenyl, 3,4,5-trihydroxyphenyl, or 1,1-biphen-4-yl. In some other embodiments, X is OSO₃⁻ and R is methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl, nonyl, ethenyl, 2-carboxyethenyl, 1-methylpropenyl, 2-methylpropenyl, 3,4,5-trihydroxyphenyl, or 1,1-biphen-4-yl. In yet some other embodiments, X is OPO₃²⁻ or SO₃⁻ and R is 2-hydroxyphenyl, 3-hydroxyphenyl or 4-hydroxyphenyl.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). An alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An “alkenyl” is an unsaturated alkyl group is one having one or more double bonds bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), and the higher homologs and isomers.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Exemplary aryl and heteroaryl groups include, but are not limited to, phenyl, 4-nitrophenyl, 1-naphthyl, 2-naphthyl, biphenyl, 4-biphenyl, pyrrole, 1-pyrrolyl, 2-pyrrolyl, 3-pyrrolyl, pyrazole, 3-pyrazolyl, imidazole, imidazolyl, 2-imidazolyl, 4-imidazolyl, benzimidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, thiazolyl, 2-thiazolyl, 4-thiazolyl, 5-thiazolyl, 2-furyl, 3-furyl, 2-thienyl, 3-thienyl, pyridine, 2-pyridyl, naphthyridinyl, 3-pyridyl, 4-pyridyl, benzophenonepyridyl, pyridazinyl, pyrazinyl, 2-pyrimidyl, 4-pyrimidyl, pyrimidinyl, 5-benzothiazolyl, purinyl, 2-benzimidazolyl, indolyl, 5-indolyl, quinoline, quinolinyl, 1-isoquinolyl, 5-isoquinolyl, 2-quinoxalinyl, 5-quinoxalinyl, 3-quinolyl, 6-quinolyl, furan, furyl or furanyl, thiophene, thiophenyl or thienyl, diphenylether, diphenylamine, and the like.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

As used herein, “fatty acid” refers to a carboxylic acid wherein R comprises a saturated or unsaturated aliphatic chain, e.g., R has the formula CₙH₂ₙ₊₁. In some embodiments of any of the aspects, the fatty acid is a monocarboxylic acid. The fatty acid can be natural or synthetic. The aliphatic chain of the fatty acid can be saturated, unsaturated, branched, straight, and/or cyclic. In some embodiments of any of the aspects, the aliphatic chain does not comprise an aromatic group. In some embodiments of any of the aspects, the aliphatic chain comprises, consists of, or consists essentially of an alkyl or alkene chain.

Exemplary carboxylic acids which are not fatty acids can include, but are not limited to lactic acid; glycolic acid; malonic acid; maleic acid; glutaric acid; citric acid; gluconic acid; and adipic acid.

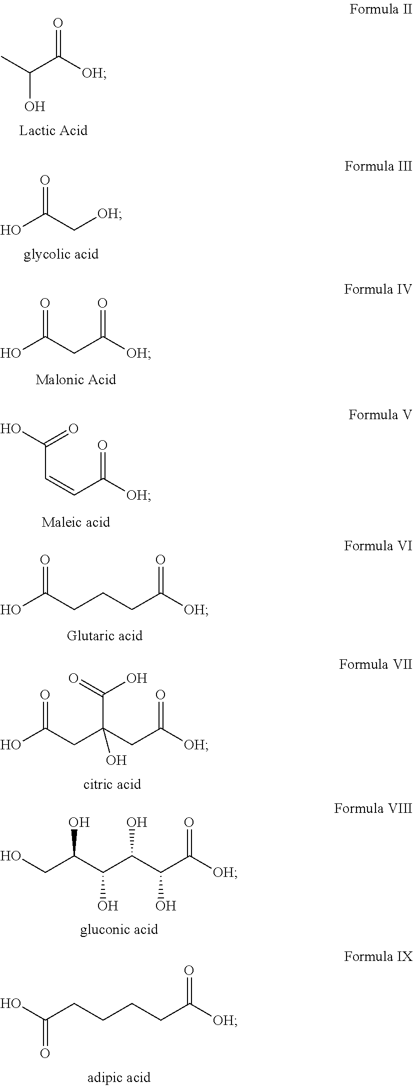

In some embodiments, the carboxylic acid which is not a fatty acid comprises no more than 5 carbons in the R group, either in a straight or branched configuration. In some embodiments, the carboxylic acid which is not a fatty acid comprises a hydroxy group in the R group. In some embodiments, the carboxylic acid which is not a fatty acid comprises one or more carboxylic acids in the R group.

In some embodiments, the carboxylic acid which is not a fatty acid comprises no more than 5 carbons in the R group, either in a straight or branched configuration, and comprises a hydroxy group in the R group. In some embodiments, the carboxylic acid which is not a fatty acid comprises 1-5 carbons in the R group, either in a straight or branched configuration, and comprises a hydroxy group in the R group.

In some embodiments, the carboxylic acid which is not a fatty acid comprises no more than 5 carbons in the R group, either in a straight or branched configuration, and comprises one or more carboxylic acid groups in the R group. In some embodiments, the carboxylic acid which is not a fatty acid comprises 1-5 carbons in the R group, either in a straight or branched configuration, and comprises one or more carboxylic acid groups in the R group.

In some embodiments, the carboxylic acid which is not a fatty acid comprises 1-5 carbons in the R group, either in a straight or branched configuration, and comprises one carboxylic acid group in the R group.

When the number of carbons in a chain is referred to herein, it is contemplated that the entire number of carbons in the chain (including branches) is referred to. In the case of a straight chain, this is the same as the carbon chain length. In the case of a branched chain, “chain length” refers to the longest carbon chain branch of the branched chain.

In some embodiments, the anion comprises one carboxylic acid group.

Exemplary carboxylic acids comprising an aliphatic chain of no more than 4 carbons can include propanoic acid (a fatty acid); isobutryic acid (a fatty acid); butyric acid (a fatty acid), 3,3-dimethylacrylic acid (a fatty acid); dimethylacrylic acid (a fatty acid); and isovaleric acid (a fatty acid).

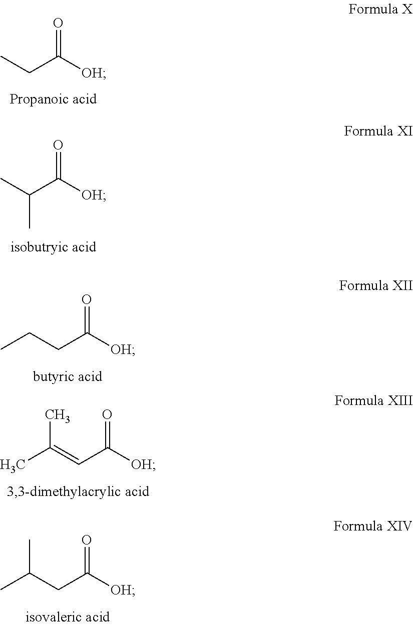

Exemplary alternative anions contemplated herein include decanoic acid and ethylhexyl sulfate.

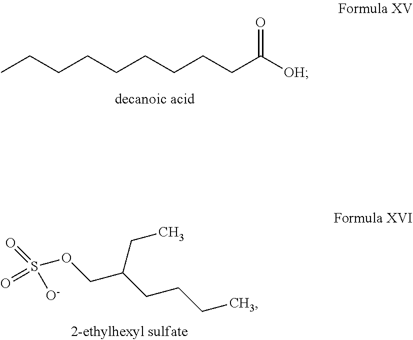

Exemplary aromatic anions include but are not limited to gallic acid, hydrocinnamic acid, hydroxybenzenesulfonic acid, 4-hydroxybenzenesulfonic acid (4-phenolsulfonic acid), biphenyl-3-carboxylic acid, and phenyl phosphoric acid.

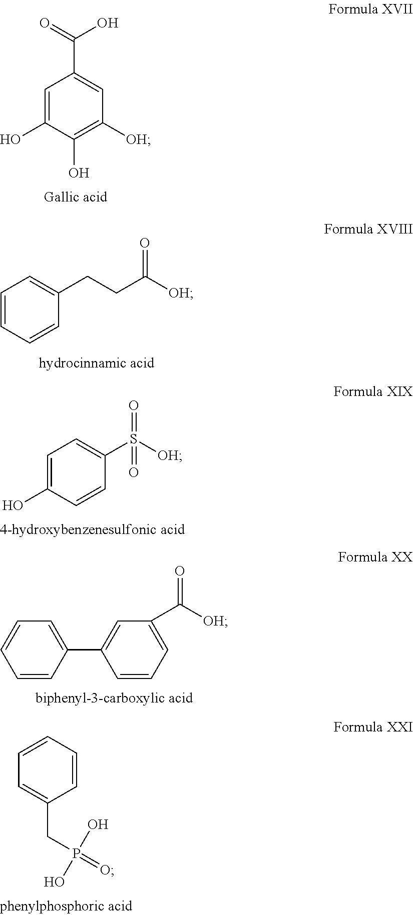 [SMILES: O=C(O)c1cc(O)c(O)c(O)c1 ; O=C(O)CCc1ccccc1 ; O=S(=O)(O)c1ccc(O)cc1 ; O=C(O)c1cccc(-c2ccccc2)c1 ; O=P(O)(O)Cc1ccccc1]

In some embodiments of any of the aspects, the anion is hydrophobic. Hydrophobicity may be assessed by analysis of log P. “Log P” refers to the logarithm of P (Partition Coefficient). P is a measure of how well a substance partitions between a lipid (oil) and water. P itself is a constant. It is defined as the ratio of concentration of compound in aqueous phase to the concentration of compound in an immiscible solvent, as the neutral molecule.

Partition Coefficient, P=[Organic]/[Aqueous] where [ ]=concentration

Log P=log₁₀(Partition Coefficient)=log₁₀ P

In practice, the Log P value will vary according to the conditions under which it is measured and the choice of partitioning solvent. A Log P value of 1 means that the concentration of the compound is ten times greater in the organic phase than in the aqueous phase. The increase in a log P value of 1 indicates a ten fold increase in the concentration of the compound in the organic phase as compared to the aqueous phase.

In some embodiments of any of the aspects, the anion has a Log P of less than 1.0. In some embodiments of any of the aspects, the anion has a Log P of less than 0.80. In some embodiments of any of the aspects, the anion has a Log P of less than 0.75. In some embodiments of any of the aspects, the anion has a Log P of less than 0.50. In some embodiments of any of the aspects, the anion has a Log P of less than 0.25. In some embodiments of any of the aspects, the anion has a Log P of less than 0.

In one aspect of any of the embodiments, the at least one ionic liquid comprises 1) an anion with a Log P of less than 1.0 and which is a carboxylic acid which is not a fatty acid, and 2) a cation comprising a quaternary ammonium. In one aspect of any of the embodiments, the at least one ionic liquid comprises 1) an anion with a Log P of less than 1.0 and which is a carboxylic acid comprising an aliphatic chain of no more than 4 carbons, and 2) a cation comprising a quaternary ammonium. In one aspect of any of the embodiments, the at least one ionic liquid comprises 1) an anion with a Log P of less than 1.0 and which is aromatic, and 2) a cation comprising a quaternary ammonium.

The Log P values for anions are known in the art and/or can be calculated by one of skill in the art. For example, PubChem and SpiderChem provide these values for various anions and chemical manufacturers typically provide them as part of the catalog listings for their products. Log P values for exemplary anions are provided in Table 1 herein.

Exemplary, non-limiting anions are provided in Table 1 below.

TABLE 1

| | LogP |
|---|---|
| Glycolic acid | −1.11 |
| Propanoic acid | 0.33 |
| Isoburtyric acid | 0.94 |
| Butyric acid | 0.79 |
| Gallic acid | 0.70 |
| Lactic acid | −0.72 |
| Malonic acid | −0.81 |
| Decanoic Acid | 4.09 |
| Maleic acid | −0.48 |
| Glutaric acid | −0.29 |
| Citric acid | −1.64 |
| 3,3-dimethylacrylic acid | 1.2 |
| Gluconic acid | −3.4 |
| Adipic acid | 0.08 |
| 2-Ethylhexyl sulfate | 3.10 |
| 4-hydroxybenzenesulfonic acid | 0.2 |
| Isovaleric acid | 1.16 |
| Hydrocinnamic acid | 1.84 |
| Phenylphosphoric acid | 1.05 |
| Biphenyl-3-carboxylic acid | 3.5 |

In some embodiments of any of the aspects, the anion is an alkane. In some embodiments of any of the aspects, the anion is an alkene. In some embodiments of any of the aspects, the anion comprises a single carboxyl group. In some embodiments of any of the aspects, the carbon chain of the carboxylic acid comprises one or more substituent groups. In some embodiments of any of the aspects, the carbon chain backbone of the carboxylic acid comprises one or more substituent groups, wherein each substituent group comprises at least one carbon atom. In some embodiments of any of the aspects, the carbon chain backbone of the carboxylic acid comprises one or more substituent groups, wherein at least one substituent group comprises a methyl group. In some embodiments of any of the aspects, the carbon chain backbone of the carboxylic acid comprises two substituent groups, wherein each substituent group comprises at least one carbon atom. In some embodiments of any of the aspects, the carbon chain backbone of the carboxylic acid comprises two substituent groups, wherein one substituent group comprises a methyl group. In some embodiments of any of the aspects, the carbon chain backbone of the carboxylic acid comprises two substituent groups, wherein each substituent group comprises a methyl group.

In some embodiments of any of the aspects, the anion is an unsubstituted alkane. In some embodiments of any of the aspects, the anion is an unsubstituted alkene. In some embodiments of any of the aspects, the carbon chain backbone of the carboxylic acid comprises one or more substituent groups. In some embodiments of any of the aspects, the carbon chain of the carboxylic acid comprises one or more substituent groups, wherein each substituent group comprises at least one carbon atom. In some embodiments of any of the aspects, the carbon chain of the carboxylic acid comprises one or more substituent groups, wherein each substituent group is alkyl, aryl, heteroalkayl, heteroaryl, alkane, or alkene. In some embodiments of any of the aspects, the carbon chain of the carboxylic acid comprises one or more substituent groups, wherein each substituent group is unsubstituted alkyl, unsubstituted aryl, unsubstituted heteroalkayl, unsubstituted heteroaryl, unsubstituted alkane, or unsubstituted alkene

In some embodiments of any of the aspects, the ionic liquid comprises a lactic acid anion.

In some embodiments of any of the aspects, the ionic liquid is choline:lactic acid (CoLa).

In some embodiments of any of the aspects, the IL is at a concentration of at least 0.01% w/v. In some embodiments of any of the aspects, the IL is at a concentration of at least 0.05% w/v. In some embodiments of any of the aspects, the IL is at a concentration of at least 0.1% w/v. In some embodiments of any of the aspects, the IL is at a concentration of at least 0.2% w/v, at least 0.3% w/v, at least 0.4% w/v, at least 0.5% w/v, at least 1% w/v or greater. In some embodiments of any of the aspects, the IL is at a concentration of from about 0.01% w/v to about 1% w/v. In some embodiments of any of the aspects, the IL is at a concentration of from 0.01% w/v to 1% w/v. In some embodiments of any of the aspects, the IL is at a concentration of from about 0.05% w/v to about 0.5% w/v. In some embodiments of any of the aspects, the IL is at a concentration of from 0.05% w/v to 0.5% w/v.

In some embodiments of any of the aspects, the IL is at a concentration of at least 25% w/w. In some embodiments of any of the aspects, the IL is at a concentration of at least 25% w/w in water. In some embodiments of any of the aspects, the IL is at a concentration of at least 25% w/w in saline or a physiologically compatible buffer.

In some embodiments of any of the aspects, the IL is at a concentration of from about 5% w/w to about 75% w/w. In some embodiments of any of the aspects, the IL is at a concentration of from 50% w/w to 750% w/w. In some embodiments of any of the aspects, the IL is at a concentration of from about 5% w/w to about 75% w/w in water, saline or a physiologically compatible buffer. In some embodiments of any of the aspects, the IL is at a concentration of from 5% w/w to 75% w/w in water, saline or a physiologically compatible buffer.

In some embodiments of any of the aspects, the IL is at a concentration of at least about 0.1% w/w. In some embodiments of any of the aspects, the IL is at a concentration of at least 0.1% w/w. In some embodiments of any of the aspects, the IL is at a concentration of from about 10% w/w to about 70% w/w. In some embodiments of any of the aspects, the IL is at a concentration of from 10% w/w to 70% w/w. In some embodiments of any of the aspects, the IL is at a concentration of from about 30% w/w to about 50% w/w. In some embodiments of any of the aspects, the IL is at a concentration of from 30% w/w to 40% w/w. In some embodiments of any of the aspects, the IL is at a concentration of from about 30% w/w to about 50% w/w. In some embodiments of any of the aspects, the IL is at a concentration of from 30% w/w to 40% w/w.

In some embodiments of any of the aspects, the % w/w concentration of the IL is % w/w concentration in water, saline, or a physiologically compatible buffer.

In some embodiments of any of the aspects, the IL is 100% by w/w or w/v.

In some embodiments, the IL is an anhydrous salt, e.g., an ionic liquid not diluted or dissolved in water. In some embodiments, the IL is provided as an aqueous solution.

In some embodiments of any of the aspects, the IL is at a concentration of at least 25% w/w and has a ratio of cation:anion of at least 1:3. In some embodiments of any of the aspects, the IL is at a concentration of at least 25% w/w in water and has a ratio of cation:anion of at least 1:3. In some embodiments of any of the aspects, the IL is at a concentration of at least 25% w/w and has a ratio of cation:anion of 1:3 or 1:4. In some embodiments of any of the aspects, the IL is at a concentration of at least 25% w/w in water and has a ratio of cation:anion of 1:3 or 1:4. In some embodiments of any of the aspects, the IL is a gel, or a shear-thining Newtonian gel.

In some embodiments of any of the aspects, the IL has a ratio of cation:anion of from about 10:1 to about 1:10. In some embodiments of any of the aspects, the IL has a ratio of cation:anion of from 10:1 to 1:10. In some embodiments of any of the aspects, the IL has a ratio of cation:anion of from about 5:1 to about 1:5. In some embodiments of any of the aspects, the IL has a ratio of cation:anion of from 5:1 to 1:5. In some embodiments of any of the aspects, the IL has a ratio of cation:anion of from about 2:1 to about 1:4. In some embodiments of any of the aspects, the IL has a ratio of cation:anion of from 2:1 to 1:4. In some embodiments of any of the aspects, the IL has a ratio of cation:anion of from about 2:1 to about 1:10. In some embodiments of any of the aspects, the IL has a ratio of cation:anion of from 2:1 to 1:10. In some embodiments of any of the aspects, the IL has a ratio of cation:anion such that there is a greater amount of anion, e.g., a ratio of less than 1:1. In some embodiments of any of the aspects, the IL has a ratio of cation:anion such that there is an excess of anion. In some embodiments of any of the aspects, the IL has a ratio of cation:anion of from about 1:1 to about 1:10. In some embodiments of any of the aspects, the IL has a ratio of cation:anion of from 1:1 to 1:10. In some embodiments of any of the aspects, the IL has a ratio of cation:anion of from about 1:1 to about 1:4. In some embodiments of any of the aspects, the IL has a ratio of cation:anion of from 1:1 to 1:4. In some embodiments of any of the aspects, the IL has a ratio of cation:anion of from about 1:1 to about 1:3. In some embodiments of any of the aspects, the IL has a ratio of cation:anion of from 1:1 to 1:3. In some embodiments of any of the aspects, the IL has a ratio of cation:anion of from about 1:1 to about 1:2. In some embodiments of any of the aspects, the IL has a ratio of cation:anion of from 1:1 to 1:2. In some embodiments of any of the aspects, the IL has a ratio of cation:anion of about 1:1, 1:2, 1:3, or 1:4. In some embodiments of any of the aspects, the IL has a ratio of cation:anion of 1:1, 1:2, 1:3, or 1:4. Without wishing to be constrained by theory, compositions with higher amounts of anion relative to cation display greater hydrophobicity.

In some embodiments of any of the aspects, e.g., when one or more nucleic acid molecules are provided in combination with the IL, the ratio of cation:anion is greater than 1:1, e.g., greater than 1:2, from about 1:2 to about 1:4, or from 1:2 to 1:4.

In some embodiments of any of the aspects, the IL is at a concentration of at least 20 mM. In some embodiments of any of the aspects, the IL is at a concentration of at least about 20 mM. In some embodiments of any of the aspects, the IL is at a concentration of at least 25 mM. In some embodiments of any of the aspects, the IL is at a concentration of at least about 25 mM. In some embodiments of any of the aspects, the IL is at a concentration of at least 50 mM. In some embodiments of any of the aspects, the IL is at a concentration of at least about 50 mM. In some embodiments of any of the aspects, the IL is at a concentration of at least 100 mM, 500 mM, 1 M, 2 M, 3 M or greater. In some embodiments of any of the aspects, the IL is at a concentration of at least about 100 mM, 500 mM, 1 M, 2 M, 3 M or greater.

In some embodiments of any of the aspects, the IL is at a concentration of from about 50 mM to about 4 M. In some embodiments of any of the aspects, the IL is at a concentration of from 50 mM to 4 M. In some embodiments of any of the aspects, the IL is at a concentration of from about 500 mM to about 4 M. In some embodiments of any of the aspects, the IL is at a concentration of from 500 mM to 4 M. In some embodiments of any of the aspects, the IL is at a concentration of from about 1 M to about 4 M. In some embodiments of any of the aspects, the IL is at a concentration of from 1 M to 4 M. In some embodiments of any of the aspects, the IL is at a concentration of from about 2 M to about 4 M. In some embodiments of any of the aspects, the IL is at a concentration of from 2 M to 4 M.

In some embodiments of any of the aspects, the IL concentration in the composition or formulation is about 0.1 mM to 20 mM. In some embodiments of any of the aspects, the IL concentration in the composition or formulation is about 0.5 mM to 20 mM, 0.5 mM to 18 mM, 0.5 mM to 16 mM, 0.5 mM to 14 mM, 0.5 mM to 12 mM, 0.5 mM to 10 mM, 0.5 mM to 8 mM, 1 mM to 20 mM, 1 mM to 18 mM, 1 mM to 16 mM, 1 mM to 14 mM, 1 mM to 12 mM, 1 mM to 10 mM, 1 mM to 8 mM, 2 mM to 20 mM, 2 mM to 18 mM, 2 mM to 16 mM, 2 mM to 14 mM, 2 mM to 12 mM, 2 mM to 10 mM, 2 mM to 8 mM, 4 mM to 20 mM, 4 mM to 18 mM, 4 mM to 16 mM, 4 mM to 14 mM, 4 mM to 12 mM, 4 mM to 10 mM, 4 mM to 8 mM, 6 mM to 20 mM, 6 mM to 18 mM, 6 mM to 14 mM, 6 mM to 12 mM, 6 mM to 10 mM, 6 mM to 8 mM, 8 mM to 20 mM, 8 mM to 18 mM, 8 mM to 16 mM, 8 mM to 14 mM, 8 mM to 12 mM, 8 mM to 10 mM, 10 mM to 20 mM, 10 mM to 18 mM, 10 mM to 16 mM, 10 mM to 14 mM, 10 mM to 12 mM, 12 mM to 20 mM, 12 mM to 18 mM, 12 mM to 16 mM, 12 mM to 14 mM, 14 mM to 20 mM, 14 mM to 18 mM, 14 mM to 16 mM, 16 mM to 20 mM, 16 mM to 18 mM, or18 mM to 20 mM. In some embodiments of any of the aspects, the IL concentration in the composition or formulation is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

As described herein, an “antigen” is a molecule that is specifically bound by a B cell receptor (BCR), T cell receptor (TCR), and/or antibody, thereby activating an immune response. An antigen can be pathogen-derived, or originate from a pathogen. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

In some embodiments of any of the aspects, a vaccine or composition described herein comprises a nucleic acid encoding an antigen.

In some embodiments of any of the aspects, the antigen can be a molecule or motif obtained or derived from a pathogen, e.g., a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, the antigen can be a molecule found in a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, the antigen can be a molecule (or antigenic portion thereof) with at least 60%, at least 65%, at least 70%, at least 75%, at least 80%, at least 85%, at least 90%, at least 95%, at least 98% or more sequence identity (nucleotide or amino acid) with a molecule found in a pathogen, e.g., a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, the antigen can be a nucleic acid encoding a protein (or antigenic portion thereof) with at least 60%, at least 65%, at least 70%, at least 75%, at least 80%, at least 85%, at least 90%, at least 95%, at least 98% or more sequence identity with a protein found in a pathogen, e.g., a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus. In some embodiments of any of the aspects, a protein with a specified sequence identity to a protein found in a pathogen retains the wild-type activity of the reference protein found in the pathogen.

In some embodiments of any of the aspects, the antigen can be a viral spike protein or antigenic portion thereof, e.g., a coronavirus or a SARS-CoV-2 virus spike protein or antigenic portion thereof. In some embodiments of any of the aspects, the antigen can be a protein with at least 60%, at least 65%, at least 70%, at least 75%, at least 80%, at least 85%, at least 90%, at least 95%, at least 98% or more sequence identity with a viral spike protein, e.g., a coronavirus or a SARS-CoV-2 virus spike protein or antigenic portion thereof.

The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals—and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the coronavirus is selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKu1. In some embodiments of any of the aspects, the coronavirus is severe acute respiratory syndrome coronavirus 2 (SARS-CoV-2), which causes coronavirus disease of 2019 (COVID19 or simply COVID). In some embodiments of any of the aspects, the coronavirus is severe acute respiratory syndrome coronavirus (SARS-CoV or SARS-CoV-1), which causes SARS. In some embodiments of any of the aspects, the coronavirus is Middle East respiratory syndrome-related coronavirus (MERS-CoV), which causes MERS.

Nucleic acids and proteins for the foregoing pathogens are known in the art, e.g., the complete genome of SARS-CoV-2 Jan. 2020/NC_045512.2 Assembly (wuhCor1) is available on the world wide web.

In some embodiments of any of the aspects, the at least one antigen is comprised by a vaccine. In some embodiments of any of the aspects, the vaccine is an attenuated vaccine. Attenuated vaccines comprise weakened or compromised versions or variants of a disease-causing microbe. Attenuated vaccines can include mutated or engineered strains of a microbe and/or strains which have been passaged in culture, thereby resulting in a loss of pathogenicity.

In some embodiments of any of the aspects, the vaccine can be a subunit vaccine, including a recombinant subunit vaccine. A subunit vaccine does not comprise entire disease-causing microbes, but only a subset of antigens obtained from or derived from the disease-causing microbe. A subunit vaccine can comprise multiple different antigens. Subunit vaccines in which the antigens are produced via recombinant technologies are termed recombinant subunit vaccines.

In some embodiments of any of the aspects, the at least one antigen is comprised by a conjugate vaccine. In conjugate vaccines, polysaccharides from a disease-causing microbe (e.g., polysaccahrides found on the surface of the microbe) are administered in combination with (e.g., conjugated to) an antigen which the patient's immune system already recognizes or which the patient's immune system will readily respond to. This increases the patient's response to the polysaccharides and provides increased protection against live versions of the disease-causing microbe. In some embodiments of any of the aspects, the antigen is a polysaccharide.

Exemplary, non-limiting vaccines suitable for use in the methods and compositions described herein can include a coronavirus vaccine; a SARS-CoV-2 vaccine; a pneumococcal vaccine; an influenza vaccine; a hepatitis B (HBV) vaccine; an acellular pertussis (aP) vaccine; a diphtheria tetanus acellular pertussis (DTaP) vaccine; a hepatitis A (HAV) vaccine; a meningococcal (MV) vaccine; and/or pneumococcal conjugate vaccine (PCV)13.

In some embodiments of any of the aspects, multiple antigens are administered. In some embodiments of any of the aspects, multiple vaccines are administered.

It is specifically contemplated that a composition or combination described herein can comprise one, two, three, or more of any of the types of components described herein. For example, a composition can comprise a mixture, solution, combination, or emulsion of two or more different ionic liquids, and/or a mixture, solution, combination, or emulsion of two or more different antigens.

As used herein, “in combination with” refers to two or more substances being present in the same formulation in any molecular or physical arrangement, e.g, in an admixture, in a solution, in a mixture, in a suspension, in a colloid, in an emulsion. The formulation can be a homogeneous or heterogenous mixture. In some embodiments of any of the aspects, the antigen can be comprised by a superstructure, e.g., nanoparticles, liposomes, vectors, cells, scaffolds, or the like, said superstructure is which in solution, mixture, admixture, suspension, etc., with the IL.

The compositions, formulations, and combinations described herein can comprise at least one IL as described herein, e.g., one IL, two ILs, three ILs, or more. In some embodiments of any of the aspects, a composition, formulation, or combination as described herein can comprise at least oLa (Choline: Lactic Acid).

The compositions and methods described herein can be administered to a subject in need of vaccination, immunization, and/or stimulation of an immune response.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an adjuvant). In some embodiments of the aspects described herein, the response is specific for a particular antigen (an “antigen-specific response”), and refers to a response by a CD4 T cell, CD8 T cell, or B cell via their antigen-specific receptor. In some embodiments of the aspects described herein, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. Stimulation of an immune response refers to an induction or increase of the immune response.

CD4+ T cells can display a Th1 or a Th2 phenotype. Pro-inflammatory CD4+ T cells are responsible for the release of inflammatory, Th1 type cytokines. Cytokines characterized as Th1 type include interleukin 2 (IL-2), γ-interferon, TNFα and IL-12. In some embodiments, cytokines characterized as Th1 type include interleukin 2 (IL-2), interferon γ, and TNFα. Such pro-inflammatory cytokines act to stimulate the immune response, in many cases resulting in the destruction of autologous tissue. Cytokines associated with suppression of T cell response are the Th2 type, and include IL-10, IL-4 and TGF-β. It has been found that Th1 and Th2 type T cells may use the identical antigen receptor in response to an immunogen; in the former producing a stimulatory response and, in the latter, a suppressive response.

In some embodiments of any of the aspects, an immune response can be an increase in or induction of a Th1 or Th2 immune response, cytokine production/release, or levels of T cells displaying a Th1 or Th2 phenotype. In some embodiments of any of the aspects, the increase is relative to the level or number in the absence of the adjuvant.

In some embodiments of any of the aspects, an immune response can be a Th1 response. In some embodiments of any of the aspects, an immune response can be cytokine production by Th1 cells. In some embodiments of any of the aspects, an immune response can be an increase in the level of Th1 antigen-specific CD4+ cells. In some embodiments of any of the aspects, an immune response can be an increase in the level of Th1 CD4+ cells. In some embodiments of any of the aspects, an immune response can be an increase in the level of Th1 cells. In some embodiments of any of the aspects, an immune response can be an increase in the level of CD4+ cells. In some embodiments of any of the aspects, the increase is relative to the level or number in the absence of the adjuvant.

In some embodiments of any of the aspects, the immune response is an increase in the IgG2a/c subclass.

In some embodiments of any of the aspects, an immune response can be an increase in activation and/or infiltration of dendritic cells. In some embodiments of any of the aspects, an immune response can be an increase in the number of and/or infiltration of CD4+ cells. In some embodiments of any of the aspects, an immune response can be an increase in the number of CD4+ cells. In some embodiments of any of the aspects, an immune response can be an increase in the infiltration of CD4+ cells. In some embodiments of any of the aspects, an immune response can be an increase in the number of and/or infiltration of Th1 CD4+ cells. In some embodiments of any of the aspects, an immune response can be an increase in the number of NK and/or CD8+ cells. In some embodiments of any of the aspects, an immune response can be an increase in the number of NK cells. In some embodiments of any of the aspects, an immune response can be an increase in the number of CD8+ cells. In some embodiments of any of the aspects, the increase is relative to the level or number in the absence of the adjuvant.

An immune response to an antigen can be the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+ T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments of any of the aspects, the methods described herein comprise administering an effective amount of compositions described herein, e.g. to a subject in order to stimulate an immune response or provide protection against the relevant pathogen the antigen was derived from. Providing protection against the relevant pathogen is stimulating the immune system such that later exposure to the antigen (e.g., on or in a live pathogen) triggers a more effective immune response than if the subject was naïve to the antigen. Protection can include faster clearance of the pathogen, reduced severity and/or time of symptoms, and/or lack of development of disease or symptoms. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or topical, administration. Administration can be local or systemic. In some embodiments of any of the aspects, the administration can be intramuscular or subcutaneous. In some embodiments of any of the aspects, the administration can be by injection, subcutaneous injection, or mucosal administration.

The term “effective amount” as used herein refers to the amount of adjuvant needed to stimulate the immune system, or in combination with an antigen, to provide a protective effect against subsequent infections, and relates to a sufficient amount of pharmacological composition to provide the desired effect. The term “therapeutically effective amount” therefore refers to an amount of the adjuvant (and optionally, the antigen) that is sufficient to provide a particular immune stimulatory effect when administered to atypical subject. An effective amount as used herein, in various contexts, would also include an amount sufficient to delay the development of a symptom of the disease, alter the course of a symptom of the disease (for example but not limited to, slowing the progression of a symptom of the disease), or prevent a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a composition which achieves a half-maximal inhibition of symptoms or induction of desired responses) as determined in cell culture, or in an appropriate animal model. Levels in plasma can be measured, for example, by high performance liquid chromatography. The effects of any particular dosage can be monitored by a suitable bioassay, e.g., assay for antibody titers, among others. The dosage can be determined by a physician and adjusted, as necessary, to suit observed effects of the treatment.

In some embodiments of any of the aspects, a therapeutically effective dose of the adjuvant and antigen comprises less antigen than a therapeutically effective dose of the antigen in the absence of the adjuvant. In some embodiments of any of the aspects, a therapeutically effective dose of the adjuvant and antigen causes a greater immune response, increased rate of an immune response, and/or greater protection than the same dose of the antigen administered without the adjuvant. In some embodiments of any of the aspects, administration of the adjuvant and antigen causes a greater immune response, increased rate of an immune response, and/or greater protection than the same dose of the antigen administered without the adjuvant.

In some embodiments of any of the aspects, the technology described herein relates to a pharmaceutical composition comprising an adjuvant as described herein, and optionally a pharmaceutically acceptable carrier. In some embodiments of any of the aspects, the active ingredients of the pharmaceutical composition comprises an adjuvant as described herein. In some embodiments of any of the aspects, the active ingredients of the pharmaceutical composition consist essentially of an adjuvant as described herein. In some embodiments of any of the aspects, the active ingredients of the pharmaceutical composition consist of an adjuvant as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent, e.g. an adjuvant as described herein.

In some embodiments of any of the aspects, a vaccine or other composition described herein can further comprise one or more adjuvants that are not or do not comprise an ionic liquid. Such adjuvants are known in the art and include, by way of non-limiting example potassium alum; potassium aluminum sulfate (Alum); aluminium hydroxide; aluminium phosphate; amorphous aluminum hydroxyphosphate sulfate (AAHS); monophosphoryl lipid A (MPLA); AS04; QS-21; MF59; CpG 1018; calcium phosphate hydroxide; paraffin oil; Adjuvant 65; Plant saponins from Quillaja, soybean, or Polygala senega; IL-1; IL-2; IL-12; Freund's complete adjuvant; Freund's incomplete adjuvant; and squalene.

In some embodiments of any of the aspects, the pharmaceutical composition comprising an adjuvant as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of an adjuvant as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an adjuvant as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the adjuvant can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the methods described herein can further comprise administering a second agent and/or treatment to the subject, e.g. as part of a combinatorial therapy.

In some embodiments of any of the aspects, an effective dose of a composition comprising an adjuvant as described herein can be administered to a patient once. In some embodiments of any of the aspects, an effective dose of a composition comprising an adjuvant can be administered to a patient repeatedly. For systemic administration, subjects can be administered a therapeutic amount of a composition comprising an adjuvant, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the adjuvant and/or the antigen. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses over a period of weeks or months.

The dosage ranges for the administration of an adjuvant according to the methods described herein depend upon, for example, the form of the adjuvant, its potency, and the extent to which symptoms, markers, or indicators of a response described herein are desired to be induced, for example the percentage inducation desired for an immune response. The dosage should not be so large as to cause adverse side effects, such as inflammatory responses. Generally, the dosage will vary with the age, condition, and sex of the patient and can be determined by one of skill in the art. The dosage can also be adjusted by the individual physician in the event of any complication.

The efficacy of the adjuvant in, e.g. to induce a response as described herein (e.g. an immune response or immunization) can be determined by the skilled clinician. However, a treatment is considered “effective treatment,” as the term is used herein, if one or more of the signs or symptoms of a condition described herein are altered in a beneficial manner, other clinically accepted signs or symptoms are improved, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Immune responses can be detected by a variety of methods known to those skilled in the art, including but not limited to, antibody production, cytotoxicity assay, proliferation assay and cytokine release assays. For example, samples of blood can be drawn from the immunized mammal and analyzed for the presence of antibodies against the antigen administered in the respective vaccine and the titer of these antibodies can be determined by methods known in the art.

Efficacy of an agent can be determined by assessing physical indicators of a desired response, (e.g. immune response, cytokine production, antibody titers, etc). It is well within the ability of one skilled in the art to monitor efficacy of administration and/or treatment by measuring any one of such parameters, or any combination of parameters. Efficacy can be assessed in animal models of a condition described herein, for example immunization of monkeys. When using an experimental animal model, efficacy of treatment is evidenced when a statistically significant change in a marker is observed.

In vitro and animal model assays are provided herein which allow the assessment of a given dose of an adjuvant and/or antigen. By way of non-limiting example, the effects of a dose of adjuvant can be assessed by measuring the antibody titers or cytokine production.

The efficacy of a given dosage combination can also be assessed in an animal model, e.g. immunization of infant or newborn monkeys as described in the Examples herein.

In one aspect of any of the embodiments, described herein is a kit comprising an adjuvant and optionally at least one antigen. In some embodiments of any of the aspects, the adjuvant and antigen are not conjugated to each other. The adjuvant and antigen can be present in the same formulation of the kit or in separate formulations of the kit, e.g., for separate administration or for mixing prior to administration.

A kit is any manufacture (e.g., a package or container) comprising at least one reagent, e.g., an adjuvant, the manufacture being promoted, distributed, or sold as a unit for performing the methods described herein. The kits described herein can optionally comprise additional components useful for performing the methods described herein. By way of example, the kit can comprise fluids and compositions (e.g., buffers, needles, syringes etc.) suitable for performing one or more of the administrations according to the methods described herein, an instructional material which describes performance of a method as described herein, and the like. Additionally, the kit may comprise an instruction leaflet.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The term “treatment” (including variations thereof, e.g., “treat” or “treated”) as used herein means any one or more of the following: (i) the prevention of infection or re-infection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In the present invention, prophylactic treatment is the preferred mode. According to a particular embodiment of the present invention, compositions and methods are provided that treat, including prophylactically and/or therapeutically immunize, a host animal against a microbial infection (e.g., a bacterium or virus). The methods of the present invention are useful for conferring prophylactic and/or therapeutic immunity to a subject. The methods of the present invention can also be practiced on subjects for biomedical research applications.

In some embodiments of any of the aspects, an immunogenic amount or immunologically effective amount of the adjuvant comprising an agonist (an optionally the antigen) is administered. The term an “immunogenic amount,” and “immunologically effective amount,” both of which are used interchangeably herein, refers to the amount of the antigen or immunogenic composition sufficient to elicit an immune response, either a cellular (T-cell) or humoral (B-cell or antibody) response, or both, as measured by standard assays known to one skilled in the art.

The term “vaccine composition” used herein is defined as composition used to elicit an immune response against an antigen within the composition in order to protect or treat an organism against disease. In some embodiments of any of the aspects, the vaccine composition is a suspension of attenuated or killed microorganisms (e.g., viruses, bacteria, or rickettsiae), or of antigenic proteins derived from them, administered for prevention, amelioration, or treatment of infectious diseases. The terms “vaccine composition” and “vaccine” are used interchangeably.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of immunization and immune response. A subject can be male or female.

A subject can be one who has been previously diagnosed with or identified as suffering from or having a condition in need of treatment (e.g. susceptibility to infection) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

As used herein, the term “corresponding to” refers to an atom or group at the specified or enumerated position in a molecule, or an atom or group that is equivalent to a specified or enumerated atom or group in a second molecule. Equivalent specified or enumerated atoms/groups can be determined by one of skill in the art, e.g., by identifying shared core structures or formulas.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method of immunizing a subject, the method comprising administering to the subject
   i) an adjuvant comprising an ionic liquid; and
   ii) at least one antigen.
2. A method of stimulating an immune response of a subject, the method comprising administering to the human an adjuvant comprising an ionic liquid.
3. The method of any of the preceding paragraphs, wherein the immune response is, or the administration results in an immune response which is, a Th1 and/or Th2 response.
4. The method of any of the preceding paragraphs, wherein the administration is by injection or mucosal administration.
5. The method of any of the preceding paragraphs, wherein the administration of the adjuvant and antigen causes a greater immune response, increased rate of an immune response, and/or greater protection than the same dose of the antigen administered without the adjuvant.
6. The method of any of the preceding paragraphs, wherein a therapeutically effective dose of the adjuvant and antigen comprises less antigen than a therapeutically effective dose of the antigen in the absence of the adjuvant.
7. A vaccine composition comprising:
   a. an adjuvant comprising an ionic liquid; and
   b. at least one antigen.
8. The method or composition of any of the preceding paragraphs, wherein the ionic liquid is choline:lactic acid (CoLa).
9. The method or composition of any of the preceding paragraphs, wherein the antigen is comprised by a vaccine selected from the group consisting of:
   a coronavirus vaccine; a SARS-CoV-2 vaccine; a pneumococcal vaccine; a hepatitis B (HBV) vaccine; an acellular pertussis (aP) vaccine; a diphtheria tetanus acellular pertussis (DTaP) vaccine; a hepatitis A (HAV) vaccine; and a meningococcal (MV) vaccine.
10. The method or composition of any of the preceding paragraphs, wherein the antigen is a molecule or motif obtained or derived from:
   a coronavirus; a SARS-CoV-2 virus; a pneumococcus; a hepatitis B virus (HBV); Bordetella pertussis; Corynebacterium diphtheria; Clostridium tetani; a hepatitis A virus (HAV); and a meningococcus.

In some embodiments, the present technology may be defined in any of the following numbered paragraphs:
1. A method of immunizing a subject, the method comprising administering to the subject
   i) an adjuvant comprising an ionic liquid; and
   ii) at least one antigen.
2. A method of stimulating an immune response of a subject, the method comprising administering to the human an adjuvant comprising an ionic liquid.
3. The method of any of the preceding paragraphs, wherein the immune response is, or the administration results in an immune response which is a Th1 and/or Th2 response.
4. The method of any of the preceding paragraphs, wherein the immune response is, or the administration results in an immune response which is an increase in Th1 and/or Th2 response as compared to the level in the absence of the adjuvant.
5. The method of any of the preceding paragraphs, wherein the immune response is, or the administration results in an immune response which is an increase in Th1 response as compared to the level in the absence of the adjuvant.
6. The method of any of the preceding paragraphs, wherein the immune response is, or the administration results in an immune response which is, an increase in activation and/or infiltration of dendritic cells as compared to the level in the absence of the adjuvant.
7. The method of any of the preceding paragraphs, wherein the immune response is, or the administration results in an immune response which is, an increase in the number and/or infiltration of CD4+ cells as compared to the level in the absence of the adjuvant.
8. The method of any of the preceding paragraphs, wherein the immune response is, or the administration results in an immune response which is, an increase in the number of NK and/or CD8+ cells as compared to the level in the absence of the adjuvant.
9. The method of any of the preceding paragraphs, wherein the administration is by injection, subcutaneous injection, or mucosal administration.
10. The method of any of the preceding paragraphs, wherein the administration of the adjuvant and antigen causes a greater immune response, increased rate of an immune response, and/or greater protection than the same dose of the antigen administered without the adjuvant.
11. The method of any of the preceding paragraphs, wherein a therapeutically effective dose of the adjuvant and antigen comprises less antigen than a therapeutically effective dose of the antigen in the absence of the adjuvant.
12. A vaccine composition comprising:
   a. an adjuvant comprising an ionic liquid; and
   b. at least one antigen.
13. The method or composition of any of the preceding paragraphs, wherein the ionic liquid comprises a quaternary ammonium cation.
14. The method or composition of any of the preceding paragraphs, wherein the ionic liquid comprises a choline cation.
15. The method or composition of any of the preceding paragraphs, wherein the ionic liquid comprises an organic acid anion.
16. The method or composition of any of the preceding paragraphs, wherein the ionic liquid comprises an organic acid anion with a log P of less than one.
17. The method or composition of any of the preceding paragraphs, wherein the ionic liquid comprises a lactic acid anion.
18. The method or composition of any of the preceding paragraphs, wherein the ionic liquid is choline:lactic acid (CoLa).
19. The method or composition of any of the preceding paragraphs, wherein the ionic liquid is at a concentration of from 1%-50% w/v.
20. The method or composition of any of the preceding paragraphs, wherein the ionic liquid is at a concentration of from 1%-30% w/v.
21. The method or composition of any of the preceding paragraphs, wherein the ionic liquid is at a concentration of from 5%-20% w/v.
22. The method or composition of any of the preceding paragraphs, wherein the ionic liquid is at a concentration of 10% w/v.
23. The method or composition of any of the preceding paragraphs, wherein the ionic liquid is an emulsion in saline.
24. The method or composition of any of the preceding paragraphs, wherein the ionic liquid has a cation:anion molar ratio of from 1:1 to 1:4.
25. The method or composition of any of the preceding paragraphs, wherein the ionic liquid has a cation:anion molar ratio of 1:2.
26. The method or composition of any of the preceding paragraphs, wherein the antigen is comprised by a vaccine selected from the group consisting of:
   a coronavirus vaccine; a SARS-CoV-2 vaccine; a pneumococcal vaccine; an influenza vaccine; a hepatitis B (HBV) vaccine; an acellular pertussis (aP) vaccine; a diphtheria tetanus acellular pertussis (DTaP) vaccine; a hepatitis A (HAV) vaccine; and a meningococcal (MV) vaccine.
27. The method or composition of any of the preceding paragraphs, wherein the antigen is a molecule or motif obtained or derived from:
   a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus.

The technology described herein is further illustrated by the following examples which in no way should be construed as being further limiting.

### EXAMPLES

### Example 1: Ionic Liquid-Based Safe Adjuvants

Adjuvants play a critical role in the design and development of novel vaccines. Despite extensive research, only a handful of vaccine adjuvants have been approved for human use comprising largely of components non-native to the human body such as aluminum salt, bacterial lipids or foreign genomic material. Described herein is the exploration of an ionic liquid-based adjuvant made using two metabolites of the body, choline and lactic acid (CoLa), that distributes the antigen efficiently upon injection, maintains antigen integrity, enhances immune infiltration at the injection site, and leads to a potent immune response against the antigen.

The current COVID-19 pandemic has brought vaccines at the forefront of medical, societal and economic challenges. Adjuvants form an important, and often essential, component of effective vaccines¹. Several materials have been explored for use as adjuvants, although only a few including aluminum salts (alum), bacterial lipids (monophosphoryl A) and foreign genome (CpG) are commonly used (See cdc.gov). A key reason for this limited translation of adjuvants is the safety concern². While a large effort is currently being focused on developing novel vaccines for COVID-19 (115 candidates as of Apr. 8, 2020)³, an alarmingly small effort is focused on developing novel adjuvants³. An effort to design better vaccines against COVID-19 and future infectious threats must include a strong effort to expand the current toolbox of adjuvants. Design of potent and safe adjuvants poses a significant challenge since they must strike a delicate balance between strong local immune stimulation and low systemic toxicity^{4, 5}. It was sought herein to address this challenge using biocompatible ionic liquids.

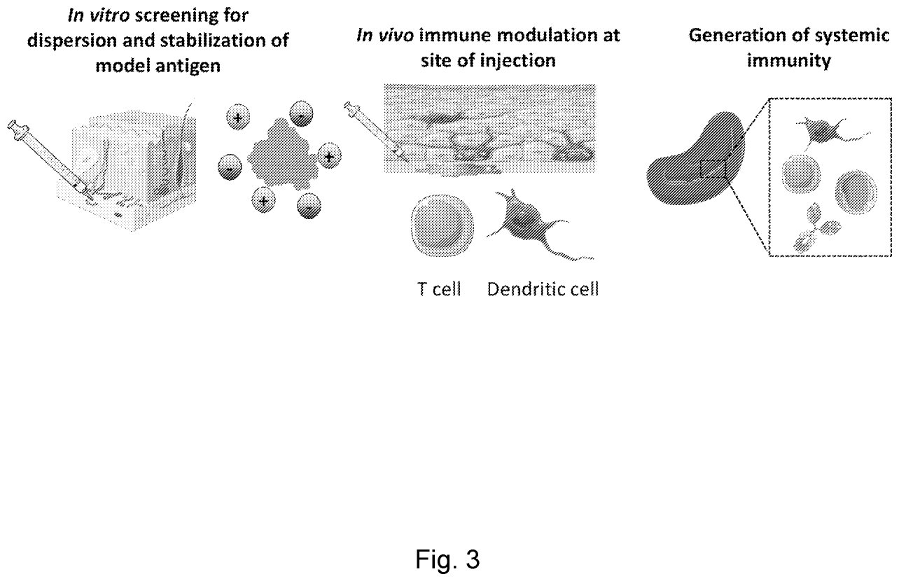

Ionic liquids and deep eutectic solvents represent a class of synthetic materials with a high degree of tunability and manufacturability⁶. They can be synthesized from components that are “generally regarded as safe” (GRAS)^{7,8}, thus improving their safety profile. ILs have been developed and used for drug delivery applications; however, their use as an adjuvant has not been yet explored. Described herein is a novel liquid adjuvant, Choline and Lactic acid (CoLa). Using ovalbumin as a model antigen, it is demonstrated that CoLa improved antigen dispersion, induced potent antigen-presenting cell (APC) infiltration at the site of injection, and generated a strong immune response against the antigen (FIG. 3).

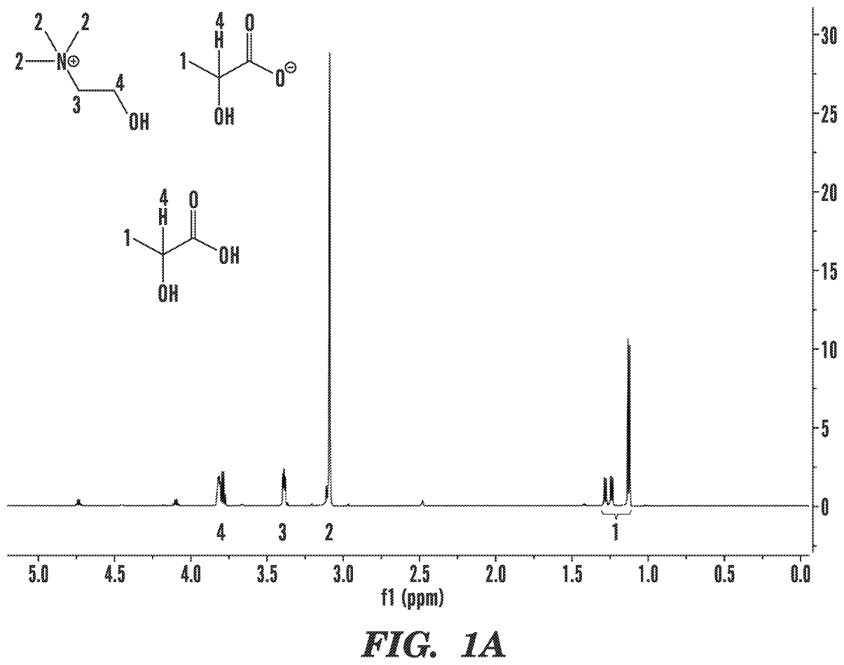

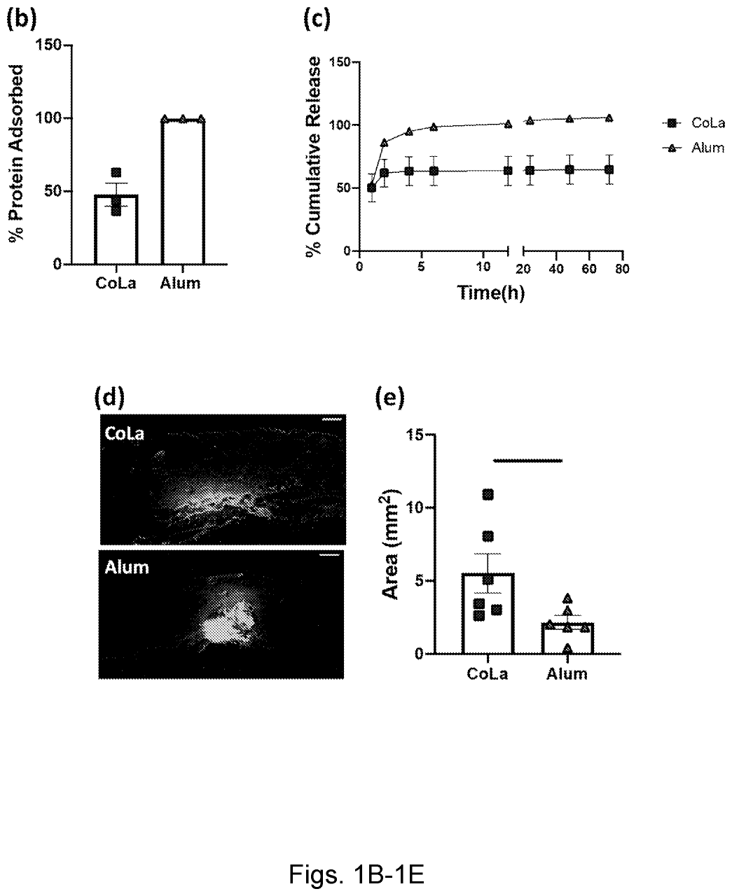

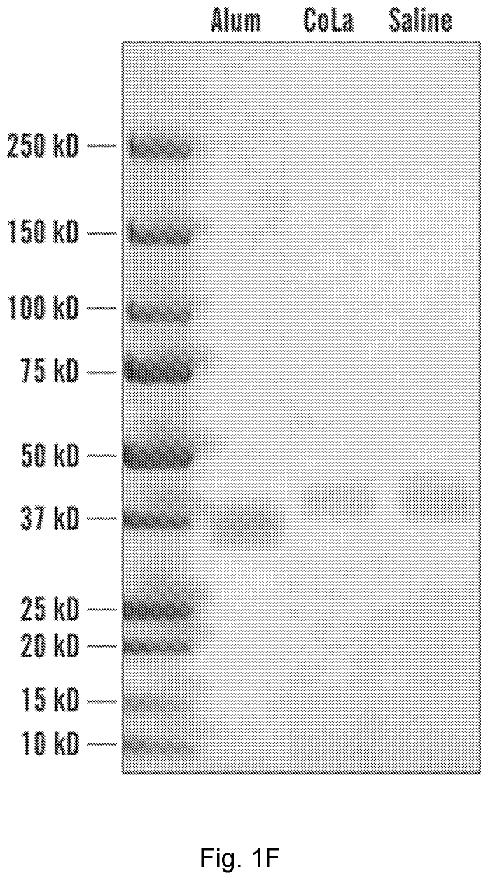

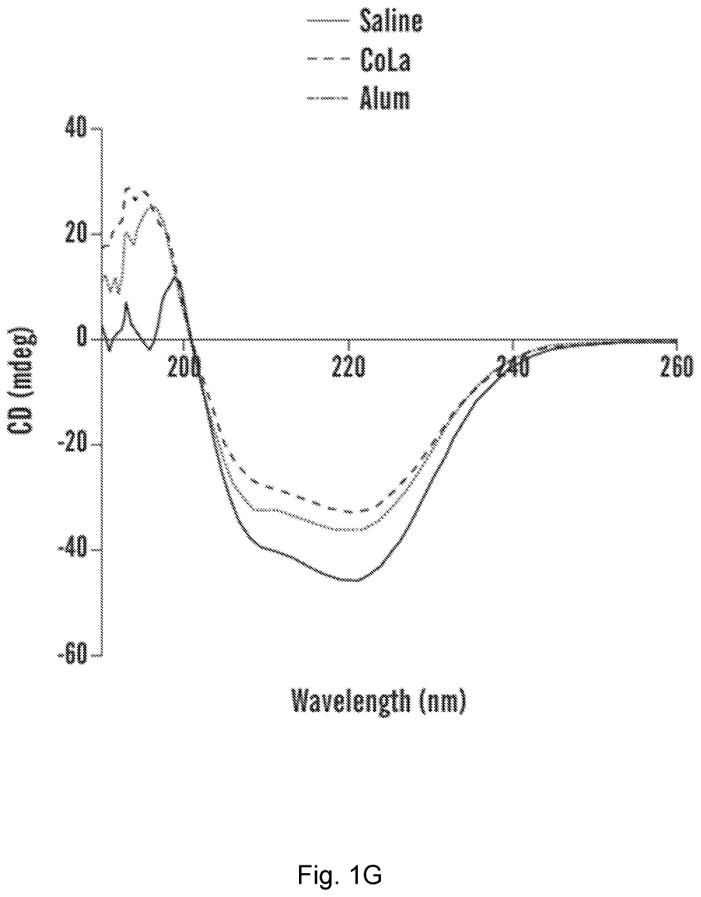

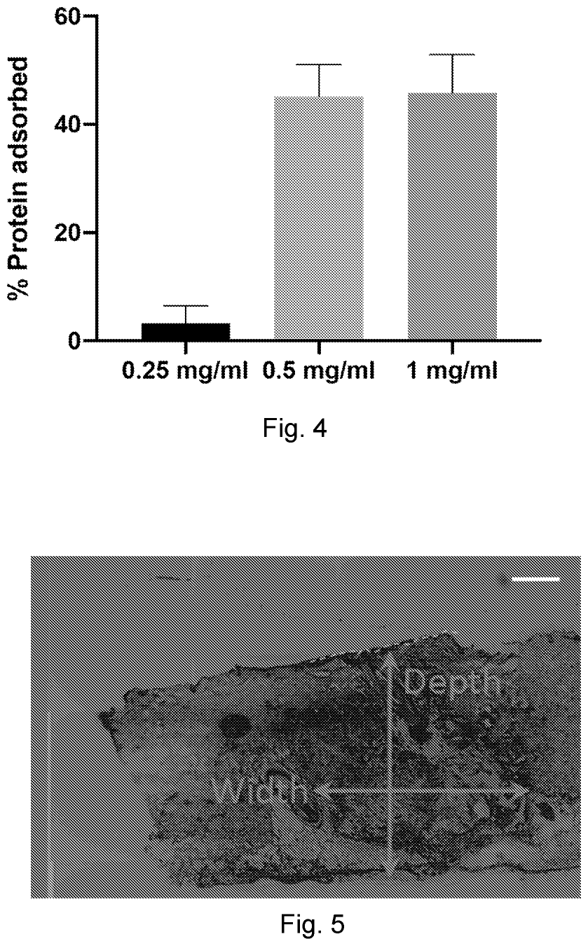

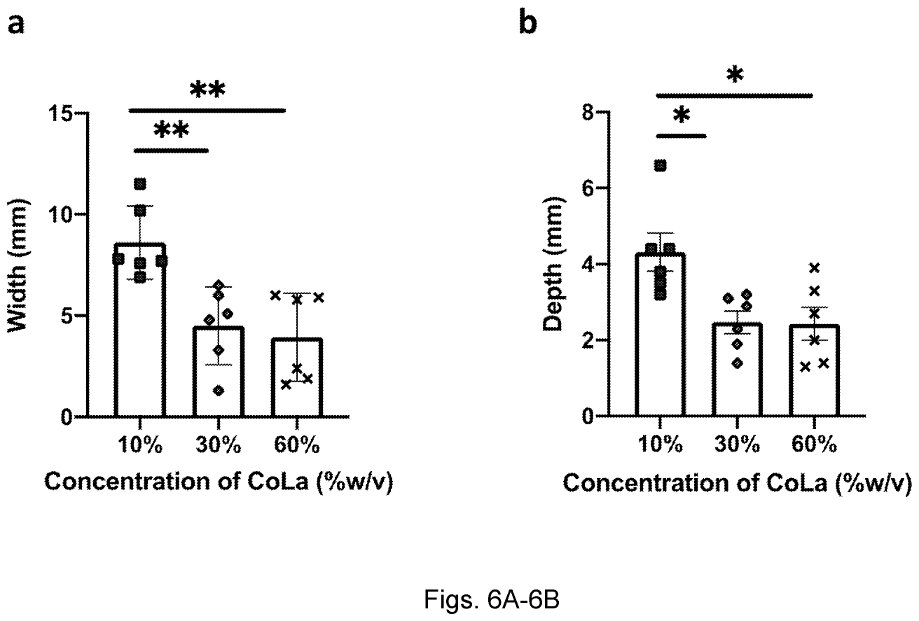

Choline and Lactic acid are natural and abundantly occurring metabolites in the human body. Further, they both have status as Generally Recognized as Safe (GRAS) molecules. CoLa (Co:La molar of 1:2) was synthesized using salt metathesis, and was verified by ¹H-NMR spectroscopy (FIG. 1A). Neat CoLa is a colorless viscous liquid which forms a milky emulsion upon dilution in saline. Upon addition to CoLa (10% w/v in saline), OVA associated with CoLa emulsion (FIG. 1B, FIG. 4) and was released over 24 h. (FIG. 1C). Compared to alum, CoLa exhibited lower adsorption and faster release (FIGS. 1B-1C). The injection of OVA-alum and OVA-CoLa in ex vivo porcine skin showed that adjuvants significantly impact antigen spreading. CoLa induced a significantly greater spread of the antigen in the skin compared to alum (FIGS. 1D, 1E, and 5). Increasing the concentration of CoLa decreased the spread, likely due to higher viscosity (FIGS. 6A-6B). SDS-PAGE indicated that CoLa maintained the molecular integrity of adsorbed OVA similar to alum and saline (FIG. 1F). CD analysis demonstrated that the secondary structure of OVA, composed majorly of a helices, is preserved by CoLa.

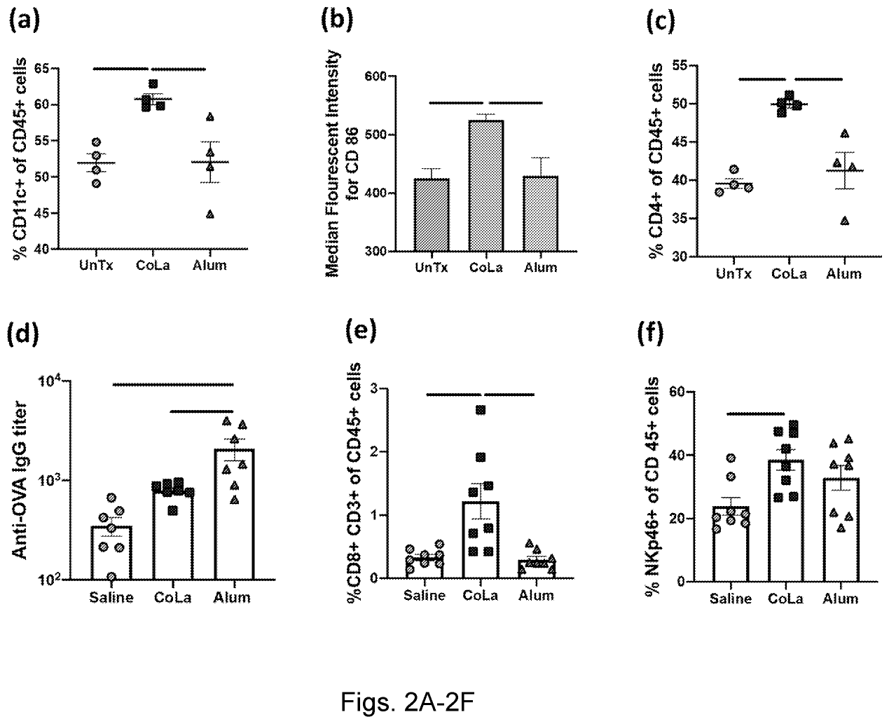

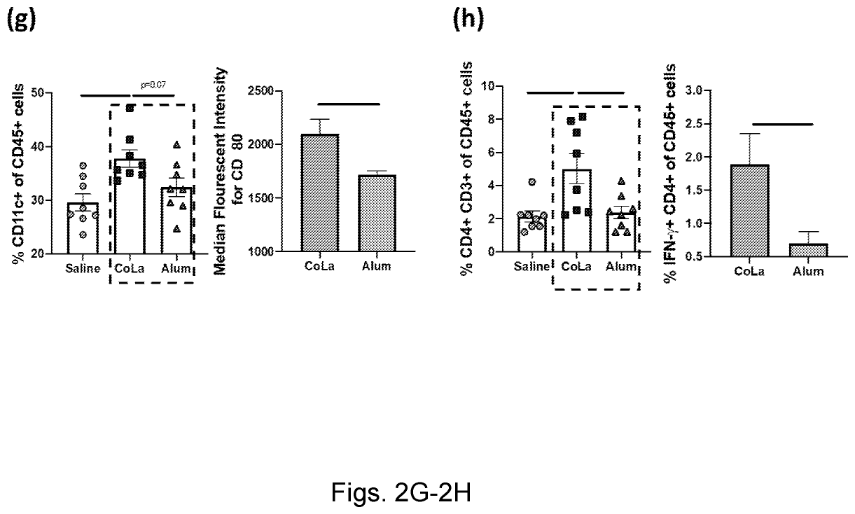

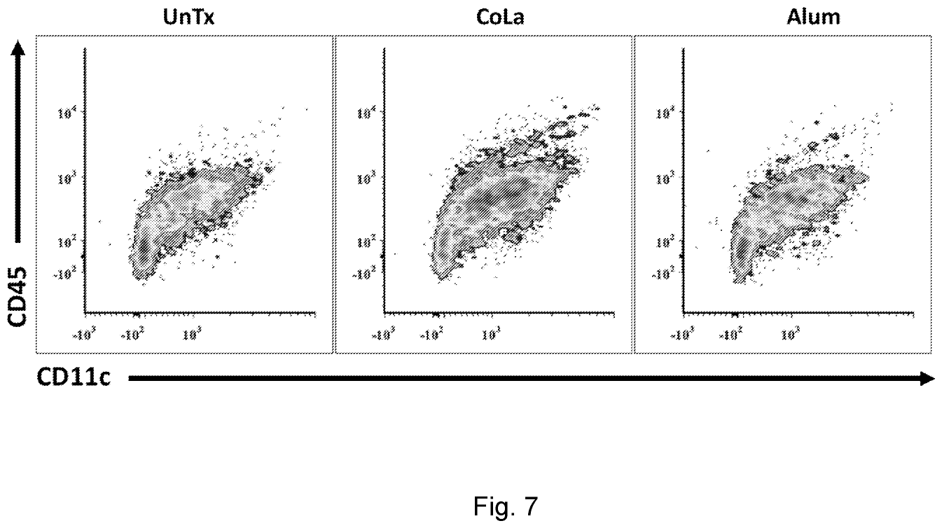

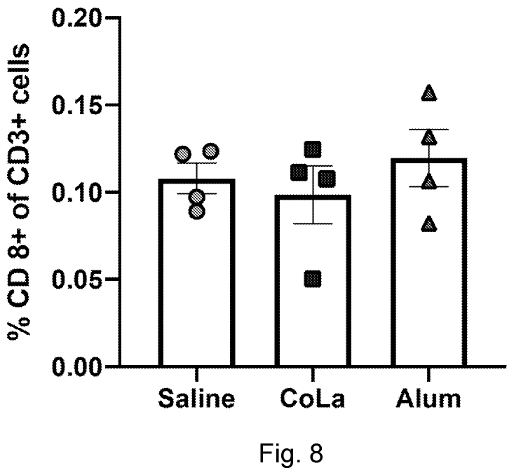

The effect of CoLa and alum on the local immune environment was assessed by subcutaneously injecting into mice and measuring the draining lymphocytes after 24 h. CoLa-treated mice showed a 20% higher infiltration of dendritic cells compared to untreated and alum-treated mice (FIG. 2A, FIG. 7). More importantly, these dendritic cells also showed a significant increase in CD86, a marker for activation, compared to the controls (FIG. 2B). Along with dendritic cells, a ˜25% increase in infiltration of CD4 cells was observed for the CoLa group, compared to the controls (FIG. 2C), indicating further antigen presentation/cross-presentation^{9, 10}, demonstrating the ability to induce a strong systemic immune response. Infiltrating CD8 cells showed no such effect (FIG. 8).

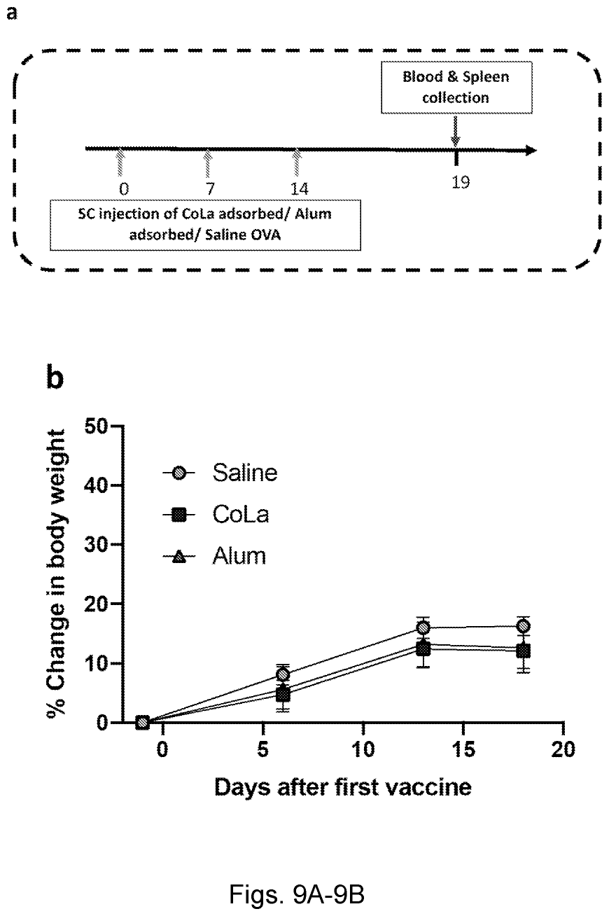

Finally, the ability of CoLa to induce immune responses was evaluated. A standard vaccination schedule was used to immunize the mice, once a week injection for a total of three weeks (FIG. 9A). In parallel, systemic toxicity of treatments was assessed by monitoring the body weight (FIG. 9B). Two-way ANOVA analysis indicated that the treatment groups minimally affected the change in body weight. Both Th1 and Th2 responses to OVA were assessed. CoLa induced a non-significant Th2 response, as assessed by anti-OVA IgG, compared to alum (FIG. 2D). On the contrary, a strong Th1 response was observed in the CoLa group. CoLa led to a 5- fold increase in the CD8 cells compared to the controls (FIG. 2E). This was accompanied by a ˜1.8-fold increase in natural killer (NK) cells compared to the saline group (FIG. 2F). CoLa group also had significantly higher activated dendritic cells (CD80) (FIG. 2G). CoLa increased the number of CD4 cells by 50% compared to both the other groups. On characterizing the CD4 population further, a ˜3-fold increase in IFN-γ+CD4 cells was observed for CoLa compared to alum (FIG. 2H). All these are markers of a potent Th1 type cellular immune response¹¹. Th1 response plays a crucial role in fight against viral infections.

The results presented here demonstrate the ability of CoLa to induce a strong Th1 immune response. CoLa and ionic liquids in general, provide a notable addition to the repertoire of available adjuvants for addressing unmet needs for protection against pandemics like COVID-19 and future infectious agent threats.

Materials and Methods

Materials

All chemicals and reagents were obtained from Sigma Aldrich and used without further purification unless otherwise mentioned. FITC-OVA was purchased from Thermo Fisher. Alhydrogel and OVA-Alexa Fluor 647 were purchased from Invitrogen. EndoFit® Ovalbumin was purchased from Invivogen. 0.9% saline solution was obtained from Teknova. Sodium phosphate buffer was purchased from Boston BioProducts. Tissue Tek OCT™ compound was obtained from Sakura Finetek. Positively charged glass slides were purchased from Fisher Scientific. Rectangular quartz cells with a 1 mm path length (1-Q-1) were obtained from Starna Cells. Laemmli protein sample buffer, 4-15% 12-well precast polyacrylamide gel, Tris/glycine/SDS running buffer, Mini-PROTEAN™ Tetra Cell Electrophoresis System, Precision Plus Protein™ All Blue Prestained Protein Standards and Bio-Safe Coomassie Stain were purchased from BioRad Laboratories. Porcine skin was obtained from Lampire Biological Laboratories. Surgical equipment was obtained from Braintree Scientific, Inc.

Synthesis and Characterization of Choline Lactate (1:2) (CoLa).

Choline bicarbonate (80% in water) was combined, with vigorous stirring, with lactic acid (85%) in a 1:2 molar ratio at 40° C. The mixture was left stirring overnight, then placed under rotary evaporation at 10 mbar and 60° C. for 2 h, before being put in a vacuum oven at 60° C. for 72 h. The resulting product was a light-yellow viscous liquid, whose chemical identity was confirmed by Nuclear Magnetic Resonance Spectroscopy. ¹HNMR (600 MHz, d-DMSO) 1.13 (dt, 3H, CH₃CH(OH)COOH); 1.27 (dt, 3H, CH₃CH(OH)COO); 4.10 (q, H, CH₃CH(OH)COOH); 4.74 (q, H, CH₃CH(OH)COO); 5.55 (bs, 3H, CH₃CH(OH)COOH; CH₃CH(OH)COO; NCH₂CH₂OH); 3.09 (s, 9H, NCH₃); 3.38 (h, 2H, NCH₂CH₂OH); 3.80 (h, 2H, NCH₂CH₂OH).

Formulation Preparation

0.5 mg mL⁻¹ ovalbumin, Alexa Fluor 647/FITC-labeled ovalbumin for in vitro experiments were dissolved in saline. For the CoLa adjuvant formulation, 10% w/v CoLa was added unless specified. Adsorbed OVA was quantified using a plate reader (Spectramax i3™)

In Vitro Drug Release Study

OVA containing solutions (0.5 mg mL-1) and complete medium (DMEM+10% FBS) were mixed to a total volume of 500 μL and incubated at 37° C. on a tube revolver. At regular time points, the suspensions were centrifuged at 12 000×g for 15 min and the supernatant was collected for analysis. The pellet was further resuspended in 400 μL fresh release media and incubated until the next time point. Samples were taken at 1, 2, 4, 6, 12, 24, 48 and 72 h after starting the incubation. The cumulative release in each release medium was quantified using OVA as fluorophore (Ex/Em 633/665) on a plate reader (Spectramax i3™)

In Vitro Dispersion

50 μL of OVA-saline (0.5 mg mL⁻¹), OVA-CoLa (0.5 mg mL⁻¹) or alum (2% suspension) were subcutaneously injected into ex vivo porcine skin. The samples were incubated for 5 h at 37° C. before frozen in optimum cutting temperature compound and sectioned into 15 μm thin slices using a cryostat (CM1950 Leica Biosystems). The tissue sections were collected on positively charged glass slides and imaged on a fluorescent microscope (Axio Zoom V16™, Zeiss). The horizontal and vertical solution diffusion throughout the skin samples (width and depth) were analyzed with the image processing software ImageJ™. Further, a MATLAB™ code developed for image processing was used to determine the surface area of the injection site¹².

Assessment of OVA Stability

An SDS-PAGE assay was carried out to assess OVA aggregation from the OVA-CoLa or alum samples. 1 mg mL-1 OVA in 10% v/v CoLa or 2% alum were incubated for 1 h at room temperature (25° C.). OVA in saline was used as a negative control. The samples were then dialyzed in 10 mM pH 7.4 sodium phosphate buffer (Boston BioProducts) for 48 h. Before electrophoresis, all samples were centrifuged at 5000×g for 5 min to discard any undissolved residue and the clear supernatants were adjusted to equivalent protein concentration. The samples were then mixed with Laemmli protein sample buffer and separated on a 4-15% 12-well precast polyacrylamide gel in Tris/glycine/SDS running buffer using a Mini-PROTEAN™ Tetra Cell Electrophoresis System (BioRad). The protein bands were stained with Bio-Safe Coomassie stain (BioRad) for observation according to the manufacturer's protocol. Circular dichroism spectrophotometry (Jasco J-1500, Easton) was performed in the far-UV region (190-250 nm) to collect spectra. The three OVA (0.5 mg mL-1) containing formulations were centrifuged for 10 min at 10,000×g. The supernatant was removed via pipetting, while the soft OVA pellet at the bottom of the tube was not disturbed. The pellet was washed with 1 mL PBS and centrifuged again to remove the supernatant. The washing/centrifugation steps were repeated until no OVA pellet was formed during centrifugation. Rectangular quartz cells with a 1 mm path length (1-Q-1) were loaded with 400 μL of a sample. As a control spectrum, OVA in PBS was used. Each spectrum was the average of three scans.

Animals

Female Balb/C mice (6-8-week-old) were purchased from Charles River Laboratories. All experiments were performed according to the approved protocols by the Institutional Animal Care and Use Committee (IACUC) of the Faculty of Arts and Sciences (FAS), Harvard University, Cambridge.

In Vivo Injection Site Modulation Studies

Balb/c mice were subcutaneously injected in the back with 50 μL of saline, CoLa or alum (n=4 for all groups). 24 h after injections, the skin from the injection site was harvested, cut into 0.5-2 mm² pieces and incubated with collagenase D (2 mg mL⁻¹), DNAse I (0.2 mg mL⁻¹), RPMI-1640 in a total volume of 5 mL PBS for 45 min at 37° C. on a tube revolver. Undigested tissue was removed by 70 μm mesh filtration. The suspension was centrifuged at 400×g for 10 min. The supernatant was removed via pipetting and 2 mL ACK lysing buffer (Thermo Fisher) was added to the pellet. After 5 min the suspensions were centrifuged again and resuspended with 2 mL FCS blocking buffer.

In Vivo Vaccination Studies.

Balb/c mice were subcutaneously injected in the back with 50 μL of OVA-saline, OVA-CoLa or alum (n=8 for all groups). A total of three injections were given on day 0, day 7 and day 14. On day 19, the mice were euthanized, blood and spleen were collected for further analysis.

Antibody Titer Measurements

Blood was centrifuged at 5000 rpm for 10 minutes at 4° C. to separate the serum from the cells. Anti-OVA IgG titer was measured as previously described¹³.

Immune Cell Profiling.

Antibody cocktails were made from CD45 (Biolegend, Cat no: 103116, Clone: 30-F11), CD 3 (Biolegend, Cat no: 100218, Clone: 17A2), CD4 (Biolegend, Cat no: 100421, Clone: GK1.5), CD8a (Biolegend, Cat no: 100711, Clone: 53-6.7), NKp46 (Biolegend, Cat no: 137606, Clone: 29A1.4), CD 11c (Biolegend, Cat no: 117307, Clone: N418), IFN-γ (Biolegend, Cat no: 505849, Clone: XMG1.2, CD86 (Biolegend, Cat no: 105011, Clone: GL-1), and Am Cyan Live/dead cell staining kit (Thermo Fischer Scientific, MA, USA). All antibodies were diluted at least 200 times prior to their use.

Statistical analyses. Statistical significance was analyzed using a two-tailed t-test, one- or two-way analysis of variance with Tukey's multiple-comparison test. p values represent different levels of significance; p<0.05 *; p<0.01 **; p<0.001 ***. Flow cytometry graphs were analyzed using FCS Express 7.O™. All data analysis was carried out with Graphpad Prism v8.O™

### REFERENCES

1. Di Pasquale, A., Preiss, S., Tavares Da Silva, F. & Garcon, N. Vaccine Adjuvants: from 1920 to 2015 and Beyond. Vaccines (Basel) 3, 320-343 (2015).
2. Kwok, R. Vaccines: The real issues in vaccine safety. Nature 473, 436-438 (2011).
3. Thanh Le, T. et al. The COVID-19 vaccine development landscape. Nat Rev Drug Discov (2020).
4. Bowen, W. S., Svrivastava, A. K., Batra, L., Barsoumian, H. & Shirwan, H. Current challenges for cancer vaccine adjuvant development. Expert Rev Vaccines 17, 207-215 (2018).
5. Petrovsky, N. Comparative Safety of Vaccine Adjuvants: A Summary of Current Evidence and Future Needs. Drug Saf38, 1059-1074 (2015).
6. Agatemor, C., Ibsen, K. N., Tanner, E. E. L. & Mitragotri, S. Ionic liquids for addressing unmet needs in healthcare. Bioeng Transl Med 3, 7-25 (2018).
7. Tanner, E. E. L. et al. Design Principles of Ionic Liquids for Transdermal Drug Delivery. Adv Mater 31, e1901103 (2019).
8. Zakrewsky, M. et al. Ionic liquids as a class of materials for transdermal delivery and pathogen neutralization. Proc Natl Acad Sci USA 111, 13313-13318 (2014).
9. Garcia Nores, G. D. et al. CD4(+) T cells are activated in regional lymph nodes and migrate to skin to initiate lymphedema. Nat Commun 9, 1970 (2018).
10. Zhao, Z., Ukidve, A., Dasgupta, A. & Mitragotri, S. Transdermal immunomodulation: Principles, advances and perspectives. Adv Drug Deliv Rev 127, 3-19 (2018).
11. Spellberg, B. & Edwards, J. E., Jr. Type 1/Type 2 immunity in infectious diseases. Clin Infect Dis 32, 76-102 (2001).
12. Cu, K., Bansal, R., Mitragotri, S. & Fernandez Rivas, D. Delivery Strategies for Skin: Comparison of Nanoliter Jets, Needles and Topical Solutions. Ann Biomed Eng (2019).
13. Zhao, Z. M. et al. Rationalization of a nanoparticle-based nicotine nanovaccine as an effective next-generation nicotine vaccine: A focus on hapten localization. Biomaterials 138, 46-56 (2017).

1. A method of immunizing a subject, the method comprising administering to the subject
i) an adjuvant comprising an ionic liquid; and
ii) at least one antigen.

2. A method of stimulating an immune response of a subject, the method comprising administering to the human an adjuvant comprising an ionic liquid.

3. (canceled)

4. The method of claim 1, wherein the the administration results in an immune response which is an increase in Th1 and/or Th2 response as compared to the level in the absence of the adjuvant.

5. The method of claim 1, wherein the administration results in an immune response which is an increase in Th1 response as compared to the level in the absence of the adjuvant.

6. The method of claim 1, wherein the administration results in an immune response which is, an increase in activation and/or infiltration of dendritic cells and/or CD4+ cells as compared to the level in the absence of the adjuvant.

7. (canceled)

8. The method of claim 1, wherein the administration results in an immune response which is, an increase in the number of NK and/or CD8+ cells as compared to the level in the absence of the adjuvant.

9. The method of claim 1, wherein the administration is by injection, subcutaneous injection, or mucosal administration.

10. The method of claim 1, wherein the administration of the adjuvant and antigen causes a greater immune response, increased rate of an immune response, and/or greater protection than the same dose of the antigen administered without the adjuvant.

11. The method of claim 1, wherein a therapeutically effective dose of the adjuvant and antigen comprises less antigen than a therapeutically effective dose of the antigen in the absence of the adjuvant.

12. A vaccine composition comprising:
i) an adjuvant comprising an ionic liquid; and
ii) at least one antigen.

13. The composition of claim 12, wherein the ionic liquid comprises a quaternary ammonium cation.

14. The composition of claim 12, wherein the ionic liquid comprises a choline cation.

15. The composition of claim 12, wherein the ionic liquid comprises an organic acid anion.

16. The composition of claim 12, wherein the ionic liquid comprises an organic acid anion with a log P of less than one.

17. The composition of claim 12, wherein the ionic liquid comprises a lactic acid anion.

18. The composition of claim 12, wherein the ionic liquid is choline:lactic acid (CoLa).

19. The composition of claim 12, wherein the ionic liquid is at a concentration of from 1%-50% w/v.

20.-23. (canceled)

24. The composition of claim 12, wherein the ionic liquid has a cation:anion molar ratio of from 1:1 to 1:4.

25. (canceled)

26. The composition of claim 12, wherein the antigen is comprised by a vaccine selected from the group consisting of a coronavirus vaccine; a SARS-CoV-2 vaccine; a pneumococcal vaccine; an influenza vaccine; a hepatitis B (HBV) vaccine; an acellular pertussis (aP) vaccine; a diphtheria tetanus acellular pertussis (DTaP) vaccine; a hepatitis A (HAV) vaccine; and a meningococcal (MV) vaccine.

27. The composition of claim 12, wherein the antigen is a molecule or motif obtained or derived from:
a coronavirus; a SARS-CoV-2 virus; a pneumococcus; an influenza virus; a hepatitis B virus (HBV); Bordetella pertussis; Corynebacterium diphtheria; Clostridium tetani; a hepatitis A virus (HAV); and a meningococcus.
